(19) United States
(12) Patent Application Publication
(10) Pub. No.: US 2026/0354794 A1
(43) Pub. Date: Nov. 12, 2026

(54) A COVER FOR AN ACOUSTIC MEDICAL DEVICE

(71) Applicant: VEINTECH PTY LTD, West Leederville (AU)

(72) Inventors: Nikhilesh BAPPOO, Wembley Downs (AU); Nicholas BUCKLEY, West Leederville (AU); Katherine ARENSON, Dianella (AU); Phuoc Hao HO, Gnangara (AU); Evan Luciano SAVIGNI, East Perth (AU); Sean Chak-Qiong FOO, Churchlands (AU); Evelyn Ciara COLLIER, Churchlands (AU); Lincoln BLACK, Kariong (AU)

(21) Appl. No.: 19/135,104

(22) PCT Filed: Dec. 6, 2023
(86) PCT No.: PCT/AU2023/051265

§ 371 Date: Jun. 3, 2025

(30) Foreign Application Priority Data
Dec. 7, 2022 (AU) 2022903739

(51) Int. Cl. A61B 8/00; A61B 50/00; A61B 50/20; A61B 90/00

(52) U.S. Cl. CPC A61B 8/4209; A61B 8/4281; A61B 8/4427; A61B 8/4444; A61B 8/4483; A61B 50/20; A61B 90/08; A61B 90/39; A61B 2050/005; A61B 2090/0808; A61B 2090/3937; A61B 2560/0214

(57) ABSTRACT

A cover for an acoustic medical device comprises a frame including a volume defined by a base and side walls extending from the base for fitting a body of the acoustic medical device within the volume. The acoustic medical device is removable from the frame which is at least partly enclosable by an acoustically conductive closure for closing the volume. The acoustically conductive closure provides a sterile barrier between the acoustic medical device and a subject.

### TECHNICAL FIELD

The present invention relates to a cover for an acoustic medical device, such as a device used for medical imaging, for example ultrasound imaging.

### BACKGROUND ART

The following discussion of the background art is intended to facilitate an understanding of the present invention only. The discussion is not an acknowledgement or admission that any of the material referred to is or was part of the common general knowledge as at the priority date of the application.

Ultrasound scanners are used in many clinical settings such as in Emergency departments, operating rooms, radiology departments and more increasingly in the pre-hospital settings.

They are used to image a wide variety of anatomical locations and are used for both diagnostic and therapeutic applications, including ultrasound guided procedures such as vascular access (Slama, M et al., (1997), Improvement of internal jugular vein cannulation using an ultrasound-guided technique, Intensive Care Medicine, 23(8), 916-919, https://doi.org/10.1007/s001340050432). Acoustic or sound waves do not penetrate air very well and so traditionally an acoustic coupling agent, typically a gel, is used between the ultrasound transducer and the area of the subject to be scanned (Casarotto, R A et al., (2004), Coupling agents in therapeutic ultrasound: acoustic and thermal behavior, Archives of Physical Medicine and Rehabilitation, 85(1), 162-165, https://doi.org/10.1016/s0003-9993(03)00293-4). This gel provides an acoustic medium that facilitates the conduction of the ultrasound waves.

When the ultrasound scanner is used in an open cavity, over broken skin, or is used to assist in a procedure where the skin barrier is breached e.g. biopsies or cannulation, the procedure needs to be performed in a sterile manner as described in local institutional guidelines (Marhofer, P & Fritsch, G, (2015), Sterile working in ultrasonography: the use of dedicated ultrasound covers and sterile ultrasound gel, Expert Review of Medical Devices, 12(6), 667-673, https://doi.org/10.1586/17434440.2015.1084872).

In most cases a form of sterile transducer cover is used. This conventionally includes a thin plastic sheath that covers the transducer and a rubber band to hold the sheath in place (Lipton, M & Ferre, R M, (2018), Ultrasound Associated Materials and Equipment. In Tayal, V S et al. (Eds.), Ultrasound Program Management: A Comprehensive Resource for Administrating Point-of-Care, Emergency, and Clinical Ultrasound, 225-241. Springer International Publishing https://doi.org/10/1007/978-3-319-63143-114). The acoustic coupling agent used with a sterile procedure also needs to be sterile (Carrico, RM et al., (2018), Ultrasound probe use and reprocessing: Results from a national survey among US infection preventionists, American Journal of Infection Control, 46(8), 913-920. https://doi.org/10.1016/j.ajic.2018.03.025). Some of the sheaths are prefilled with the sterile coupling agent, others use prefilled sterile acoustic coupling agent sachets. Other covers are more rigid and snap over the transducer. When a sterile cover is used, the acoustic coupling agent needs to be applied between the transducer and the sterile cover as well as between the sterile cover and the subject's skin (Marhofer & Fritsch, (2015)).

Ultrasound probes are not required to be sterilised between each subject as long as the skin over the scanned area is intact. The transducer is typically cleaned with soapy water or an antiseptic solution. However, after a transducer has been used over an open wound or for a procedure where the skin is punctured, the transducer should be sterilised (American Institute of Ultrasound in Medicine, (2016), Guidelines for cleaning and preparing external-and internal-use ultrasound probes between patients, AIUM Practice Guidelines). Transducers cannot be autoclaved or gas sterilised. They can be sterilised by placing them in a Trophon device for the manufacturer's specified amount of time, or using an anti-bacterial, anti-fungal and anti-sporicidal multiple wipe system e.g. the Trisel system. Present cleaning practice guidelines are not universal and often poorly adhered to, leading to the risk of cross contamination from contaminated probes (Carrico et al., (2018)).

The current sterile sheaths are cumbersome and difficult to use, often requiring two operators to ensure full sterility (aseptic technique) while fitting the sheath over the transducer. The probe is non-sterile, while the sheath is sterile, meaning that the operator cannot handle the probe himself or herself to place in the probe sheath while wearing sterile gloves. The sheaths are typically around a metre in length and poorly fitting around the transducer. This creates difficulty in ensuring that the transducer is well covered with gel which is necessary to prevent air bubbles degrading the ultrasound image.

The present invention has been developed against the above background.

### SUMMARY OF INVENTION

In one aspect, the present invention provides a cover for an acoustic medical device comprising a frame including a volume defined by a base and side walls extending from the base for fitting a body of the acoustic medical device within the volume, said acoustic medical device being removable from said frame; and wherein the frame is at least partly enclosable by an acoustically conductive closure for closing the volume, the acoustically conductive closure providing a sterile barrier between the acoustic medical device and a subject.

The frame may include or comprise an acoustically conductive portion with the frame providing a sterile barrier between the acoustic medical device and a subject.

Advantageously, the acoustically conductive closure allows for a medical procedure to be performed in sterile manner without requirement for sterilising the acoustic medical device which can be problematic. The frame conveniently also enables the retention of the acoustic medical device without a user needing to touch the acoustic medical device. The frame may be configured, for example by flexing or stretching, to fit a range of acoustic medical devices, for example for ultrasound, whether wired or wireless.

The side walls may extend around all sides of the frame. However, the side walls may be provided as a shorter wall or as alignment guides extending from a plurality of sides of the base, for example two opposed sides of the frame, particularly where this allows a preferred frictional fitting of acoustic medical device to the frame. It will be understood that other arrangements of base and side walls or alignment guides are possible.

The frame may comprise a plurality of portions, for example one being the base or bottom frame portion and another being the top frame portion. The base can be adhered or otherwise connected to the further portion of the frame by connection means, conveniently of tongue and groove type, though alternative clip or snap fit connection means could be used in other embodiments. A cover formed by a plurality of portions may have a range of configurations and shapes, for example in the shape of a strawberry punnet.

The closure allows for disposal and replacement enabling a new closure, conveniently of the same kind, to be used for each procedure utilising the acoustic medical device. Conveniently, the closure is formable from a sheet, optionally of a polymer film. Such a polymer film closure may be described as a polymer sheath and it is conveniently transparent to allow convenient operation of the acoustic medical device. The closure may envelop the acoustic medical device and the frame facilitating operation of the acoustic medical device without direct manual contact with it. The closure may be stretched to fit the frame or cover.

The polymer film may be cut to shape for fitting to the frame and may include elements, such as a plurality of flaps or tabs to facilitate fitting to the frame. The polymer film could be formed as a sleeve, envelope or pocket enclosing the frame and acoustic medical device so as to provide the barrier described above. The removable closure, conveniently of polymer film, is connected to the housing by adhesive, desirably easily removable adhesive, which may be disposed on flaps or tabs as adhesive strips or other convenient arrangements of adhesive. The frame may comprise peelable layer(s) which may be removed when the cover is used, conveniently to maintain sterility up to use in a procedure. The cover could be supplied in this form to save on packaging and consequential waste.

An alternative closure may take the form of a lid that is opened to allow accommodation of the acoustic medical device within the frame and closed afterwards. The lid could be either hinged to the frame or detachable from the frame. The lid, whether connected to or detachable from the frame, may be a flexible film or made of a rigid inflexible material.

The frame may be fitted to the body of the acoustic medical device in a number of ways including frictional grip and adhesion. The frame could be rigid and inflexible but may also be of a flexible material that allows a frictional fit of the acoustic medical device within the frame. The frame may, in some embodiments, be conformable to the shape of the acoustic medical device but of slightly lesser dimension such that flexing of the frame, or frame portions, such as the connection means described above, is required to accommodate the acoustic medical device. A frictional grip then retains the body of the acoustic medical device within the cover allowing effective integration of cover with acoustic medical device. This minimises the possibility of air gaps that can interfere with the procedure by creating noise on an acoustic signal required for accurate operation of the acoustic medical device and allows collection of higher quality data from the acoustic medical device.

The frame may include alignment guides for the acoustic medical device to facilitate friction fitting of the acoustic medical device within the frame and minimising or eliminating movement of acoustic medical device within the frame. Connecting means that include holes or sharp edges are desirably avoided as these could cause cleaning difficulties.

Conveniently, the frame accommodates an acoustic coupling medium for reducing acoustic impedance between the acoustic medical device and the closure, conveniently the polymer sheath. For example, a base of the frame may, for example, include a compartment or pocket for containment of acoustic coupling medium. The pocket may be formed in a range of ways, for example form-fill-seal. The acoustic coupling medium may be contained within a matrix, such as a mesh with pore sizes below 1-2 microns to prevent the migration of bacteria. Conveniently, the pocket contains the acoustic coupling medium to a specific area, for example proximate the transducer array in an ultrasound probe. In one embodiment, the cover may include the transducer array. In that case, signals may be transmitted from the transducer array to the acoustic medical device by wire or wireless communications network. In that regard, the cover may be applied to both wired and wireless acoustic medical devices.

Alternatively, the frame may be communicable with a receptacle or reservoir for acoustic coupling medium. Such a receptacle could include an acoustic coupling medium filled syringe co-operable with the cover to apply the acoustic coupling medium, advantageously automatically. The compartment or pocket for acoustic coupling medium may further comprise a removable cover or peelable layer, preferably a thin film or membrane, which may be peeled off the base, either internally or externally of the cover, to expose the acoustic coupling medium, such as a gel or deformable solid acoustically transmissive hydrogel. In the case of an acoustically conductive hydrogel, the hydrogel could have properties tuneable by an external energy source such as, but not limited to: heat, mechanical energy, electrical energy or acoustic energy. The peelable layer may be provided with a tab to ease removal. Where multiple peelable layers are provided, these may be provided with indicia which inform a user of the order in which the peelable layers should be removed.

The thin film or membrane conveniently comprises a plurality of pores or openings to allow passage of excess acoustic coupling medium. Such passage may involve expression or extrusion. Compression of the acoustic cover conveniently causes passage or expression of the acoustic coupling medium through the plurality of pores or openings.

The capacity of the cover to hold and, preferably, provide acoustic coupling medium makes it both compatible with, and complementary to, an acoustic medical procedure, in particular an ultrasound procedure. The compartment or pocket desirably contains a volume of acoustic coupling medium, such as medical grade ultrasound gel, to enable effective acoustic coupling sufficient to reduce signal noise or loss while maintaining the sterile field required for the medical procedure. For example, the volume of an ultrasound gel in the compartment or pocket is preferably less than 120 cm³, more preferably 110 cm³ or 100 cm³, most preferably less than 90 cm³, for example less than 50 cm³, for example less than 30 cm³.

The frame or cover may be low profile, not covering all of the acoustic medical device but rather a proportion thereof. For example, the frame or cover may cover less than 90% of the height of the side walls, more preferably less than 50% of the side walls and most preferably less than 30% of the height of the side walls. A low profile reduces the amount of material required for the frame which, where plastic, provides greater environmental sustainability.

The frame or cover, for example a two part cover as described above, is conveniently provided with an ergonomically effective configuration so that most users find the frame or cover easy to hold and comfortable to use. To this end, holding means such as extending rims, grooves or indentations may be provided to guide a user's hand or fingers. Such holding means may also be located to prevent hand contact with areas of operational importance from the perspectives of either maintaining sterility, preventing obstruction of energy beams and so on. The ergonomically effective configuration allows ease of use of the acoustic medical device which in turn allows more precise conduct of a procedure such as cannulation for a wide range of cannulas in particular when taken in combination with effective acoustic coupling as described above.

The cover may comprise a needle guide for guiding placement of a needle or needle-like object, e.g. a cannula, lumbar puncture needle or surgical guide, to be used in a medical procedure. The needle guide may include means, such as a tube or notch, for restricting movement along the plane of insertion of the needle or needle-like object. Where the needle guide is a tube, it may be set upon an axis allowing rotation of the tube and so the needle guide. Such a needle guide would be advantageous where the placement of a needle at a selected angle is required by the procedure, for example as in cannulation (using cannulas such as from Becton Dickinson (e.g. BD Venflon Pro Safety Needle Protected IV cannula, BD Insyte™ Autoguard™ Shielded IV Catheter and BD Nexiva™ Diffusics™ Closed IV Catheter system), BBraun (e.g. Introcan Safety and Vasofix Safety) and Teleflex (e.g. Arrow series cannulas). The needle guide could be automated with potential advantages in terms of workflow and efficiency of the procedure while also ensuring a sterile field by minimising or removing user error. Other forms of needle guide could be employed. For example, the needle guide could be electronic, for example being in the form of an LED or other device that focusses a desired location for placement of a needle or cannula.

The cover desirably includes a connection means, whether connected to the frame or the removable closure, for detachably connecting the cover to a subject, such as a patient. This facilitates hands-free operation of the acoustic medical device and avoidance of the need for a user to manually hold both the cover and the acoustic medical device in position. The user can rather have both hands available to operate the acoustic medical device. The connection means may be configured to a range of anatomical locations, in any event facilitating hands free operation of the acoustic medical device.

The connection means may comprise fastening straps which may be fastened to a subject, for example to a limb of the subject. The limb may be a forearm of a human subject. The connection means is preferably provided with adjustment means to allow fitting to a range of subjects. The connection means may act as a tourniquet dependent on the nature of the procedure, e.g. a tourniquet is required for cannulation or venipuncture. A pressure sensor may be included with such fastening straps to indicate pressure, in particular in cases where pressure is excessive or insufficient for the required purpose.

Conveniently, the acoustic medical device is a portable acoustic medical imaging device, such as an ultrasound probe, though the cover could be used for other medical imaging devices using radiation in other forms including as visible, ultraviolet or infra-red light. Such acoustic medical devices may conveniently be used in procedures requiring sterility, for example an ultrasound procedure such as venipuncture or cannulation. The term ‘acoustic medical device’ may also be applied to devices for veterinary use.

The cover may be electrically or electro-mechanically configured—for example through use of RFID, mechanical switches or the Hall Effect—to cause the activation of the acoustic medical device, conveniently upon accommodation of the acoustic medical device within the frame. Activation of the acoustic medical device serves as an indicator that a sterile field is to be maintained to enable safe performance of the medical procedure.

The cover may be provided with functionality otherwise provided on the acoustic device either to encourage or force use of the cover or for convenience of design or use of the acoustic device. For example, the cover may be provided with a battery or other power supply for the acoustic device which cannot be accessed unless the cover is correctly positioned to enclose or otherwise appropriately cover the device for use in a sterile procedure.

The cover could also be configured to provide a display for the acoustic device. Such configuration may include providing optical features such as a polarising filter or an in-built screen. The polarising filter may be required to enable viewing of the screen thus encouraging or forcing use of the cover. Similarly, where the cover includes an in-built screen, a user may be prevented from viewing data from the acoustic device unless the cover is appropriately placed on or over the acoustic device. An example would be the prevention of visualisation of sub-cutaneous structures using an acoustic medical device during cannulation. Advantageously the user would be required to set up a sterile field using the sterile sheath to allow the visualisation of sub-cutaneous structures.

The cover may include means for controlling beam direction. For example, a membrane placed proximate the transducer array may be configured as a concave or convex lens to diverge or converge an ultrasound (or other) energy beam. Such a membrane may be oriented at an angle to direct a beam at a desired angle or the membrane may simply allow direct transmission of energy. The inclusion of controlled beam directions, advantageously allows focusing of energy beam to produce a better resolution for medical imaging and higher transfer of acoustic energy for therapeutic applications (such as but not limited to physiotherapy applications of high intensity frequency ultrasound).

In a further aspect, the present invention provides a method for using the cover in a procedure comprising:
providing a frame including a volume defined by a base and side walls extending from the base;
fitting the acoustic medical device within the volume, said acoustic medical device being removable from said frame;
enclosing the frame and the acoustic medical device by closing the volume by an acoustically transparent closure, the acoustically transparent closure providing a sterile barrier between the acoustic medical device and a subject; and
conducting the procedure.

In a still further aspect, the present invention provides a method for fabricating the cover. As described above, certain embodiments require manufacture of a cover assembled both solid and liquid components. Preferably, both solid and liquid components are manufactured at the same time. In this case, preferred temperature for manufacture would be below 20 °C, more preferably below 15 °C, most preferably below 10 °C or 5 °C.

In a still further aspect, the present invention provides a dispenser for dispensing covers or frame portions such as those described above. The dispenser stores covers or frame portions which may be dispensed by a spring loaded or other mechanism as required by a user.

In a still further aspect, the present invention provides a cover for an acoustic medical device comprising an elongate elastic portion and a relatively inelastic or rigid portion and a source of acoustic couplant. Such a cover may be stretched to cover a range of acoustic device geometries while providing acoustic couplant to the required surfaces. Conveniently, the cover is provided with a receptacle provided with a base and a post vertically extending from the base from which the cover is stretched to fit the acoustic device.

In a still further aspect, the present invention provides a cover for an acoustic medical device configured as an acoustically transparent dressing. Such a cover may be used with a range of acoustic medical devices.

In a still further aspect, the present invention provides a sleeve allowing provision of a sterile barrier between patient, for example a patient's limb, and an acoustic medical device. Such a sleeve may be used with a cover as described above or separately.

Among a range of advantages, the above-described cover enables aseptic operation by a single person without a specific specialisation. Conveniently, the cover may be used to assist sterile venipuncture or cannulation and other ultrasonic imaging of sub-cutaneous structures by a wider range of personnel without formal specialised training including medical and paramedical professionals such as, but not limited to, registered nurses, laboratory phlebotomists, therapists and researchers. A range of examples is described below. The cover can be deployed in a manner that avoids direct contact of the acoustic medical device with a subject and which allows a user to aseptically cover the acoustic medical device without touching it.

### BRIEF DESCRIPTION OF THE DRAWINGS

Further features of the acoustic conductive cover of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a diagrammatic representation of an acoustically conductive cover for a medical imaging device, being an ultrasound probe, according to one embodiment of the present invention.

FIG. 2 is a diagrammatic representation of the acoustically conductive cover of FIG. 1.

FIG. 3 is a cross-sectional view of the acoustically conductive cover of FIGS. 1 and 2 covering the ultrasound probe.

FIG. 4 is a front view of the acoustically conductive cover of FIGS. 1 to 3.

FIG. 5 is a cross-sectional view of the acoustically conductive cover of FIGS. 1 to 4 showing the containment of the acoustic coupling medium.

FIG. 6 is a diagrammatic representation of the acoustic conductive cover of FIGS. 1 to 5 including a polymer sheath with adhesive tabs to cover an acoustic medical device.

FIG. 7 is a diagrammatic representation of a second embodiment of an acoustically conductive cover stretchable over an acoustic medical device.

FIG. 8 is a flowchart outlining the workflow for use of the acoustic transmissive cover as shown in FIGS. 1 to 7 during a medical procedure with an acoustic medical device deployed with the acoustic transmissive cover.

FIGS. 9(a) and 9(b) are respective diagrammatic representations for a cover housing a wireless ultrasound probe and a conventional wired ultrasound probe.

FIGS. 10(a) and 10(b) are respective diagrammatic representations of alternative configurations for a gel containing receptacle for the acoustically conductive cover of embodiments of the invention.

FIGS. 11(a) and 11(b) are diagrammatic representations showing a further embodiment of frame of the acoustically conductive cover of the present invention in which the frame comprises two connectable portions.

FIGS. 12(a) and 12(b) are orthogonal views showing the bottom frame portion of a two part cover comprising a bottom frame portion and a top frame portion according to a fifth embodiment of the present invention.

FIG. 13 is an orthogonal view of a top frame portion to be fitted to a bottom frame portion which may be fitted to the bottom frame portion of FIGS. 12(a) and (b).

FIG. 14 is an orthogonal view of the cover formed from fitting the bottom frame portion of FIG. 12(a) or 12(b) to the top frame portion of FIG. 13.

FIG. 15 is an orthogonal view of a two part cover formed from a bottom frame portion of FIG. 12(a) or 12(b) hingedly connected to the top frame portion of FIG. 13 according to a sixth embodiment.

FIG. 15A is an orthogonal view of the cover of FIG. 14 showing a vein path through an optically transparent portion of the top frame portion.

FIG. 15B is a schematic cross-section of the cover of FIG. 15A showing connection between an acoustic medical device and the top frame portion.

FIG. 16(a) is an orthogonal view of a bottom frame portion for a two part cover according to a seventh embodiment.

FIG. 16(b) is a side section view of the bottom frame portion of FIG. 16(a).

FIG. 16(c) is a side section view of a top frame portion which is fitted to the bottom frame portion of FIGS. 16(a) and (b) to form the cover of the seventh embodiment.

FIGS. 17 to 19 schematically show different mechanisms for fitting bottom and top frame portions together to form a two part cover such as that shown in FIGS. 16(a) to (c).

FIG. 20 schematically shows portion of a two part cover, such as those of the sixth and seventh embodiments, configured for ergonomic holding by a user.

FIG. 21 is a bottom orthogonal view of a bottom frame portion to be fitted to a top frame portion to form a two part cover of an eighth embodiment and including peelable tabs to facilitate use of the cover.

FIG. 22 is a top orthogonal view of the bottom frame portion of FIG. 21.

FIG. 23 is a top orthogonal view of the bottom frame portion of FIG. 22 with a first alternative arrangement of peelable tabs.

FIG. 24 is a top orthogonal view of the bottom frame portion of FIG. 22 with a second alternative arrangement of peelable tabs.

FIGS. 25(a) and (b) show top orthogonal views of a bottom frame portion for a two part cover according to a ninth embodiment in which the cover is configured for supplying battery power to an acoustic medical device.

FIGS. 26(a) and (b) show top orthogonal views of a top frame portion for a two part cover according to a tenth embodiment in which the top frame portion includes a polarising filter to enable viewing of the screen of an acoustic medical device.

FIGS. 27(a) and (b) are top orthogonal views of a top frame portion for a two part cover according to an eleventh embodiment in which the top frame portion includes an in-built screen to display data from the acoustic medical device.

FIGS. 28(a) to (d) are schematic views illustrating control over direction of an emitted ultrasound beam by configuration of a membrane placed adjacent transducers of the acoustic medical device according to a twelfth embodiment.

FIGS. 29(a) and (b) show schematic views of a dispenser for dispensing cover portions for use in forming a sterile cover according to a thirteenth embodiment.

FIG. 30 is an orthogonal view of a stretchable cover for an acoustic medical device according to a fourteenth embodiment.

FIG. 31 is an orthogonal view of a receptacle for holding the stretchable cover of FIG. 30 showing a post for locating the stretchable cover which is omitted for purposes of illustration.

FIG. 32 is an orthogonal view of the receptacle of FIG. 31 with stretchable cover in position prior to use to cover the acoustic medical device.

FIG. 33 is a schematic view showing removal, for use, of the stretchable cover of FIGS. 30 and 32 from the receptacle of FIGS. 31 and 32.

FIG. 34 is a flowchart outlining the workflow for use of the stretchable cover as shown in FIGS. 30, 32 and 33 during a medical procedure with an acoustic medical device deployed with the acoustic transmissive cover.

FIG. 35(a) is a flowchart outlining the workflow for use, in a sterile procedure, of a two part cover, such as the two part cover of the fifth to eleventh embodiments, during a medical procedure with an acoustic medical device deployed with the acoustic transmissive cover.

FIG. 35(b) is a flowchart outlining the workflow for use, in a clean procedure, of a two part cover, such as the two part cover of the fifth to eleventh embodiments, during a medical or veterinary procedure with an acoustic device deployed with the acoustic transmissive cover.

FIG. 36 is a schematic plan view of a cover for an acoustic medical device, the cover being provided as an adhesive dressing according to a fifteenth embodiment.

FIG. 37 is a schematic side view of the cover of FIG. 36.

FIGS. 38(a) and (b) show schematic views of a sleeve acting as a cover for an acoustic device according to a sixteenth embodiment.

### DESCRIPTION OF PREFERRED EMBODIMENTS

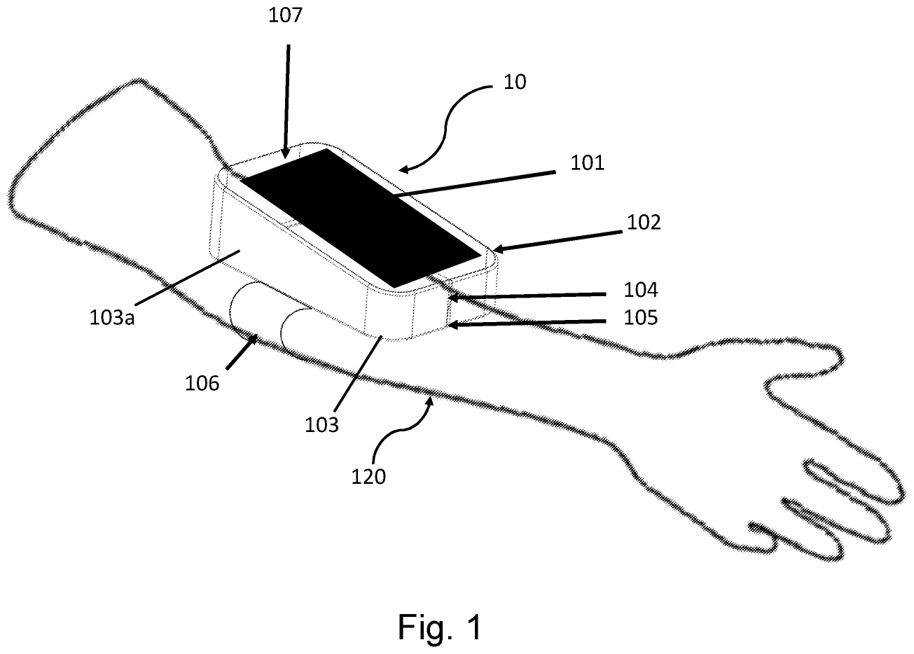

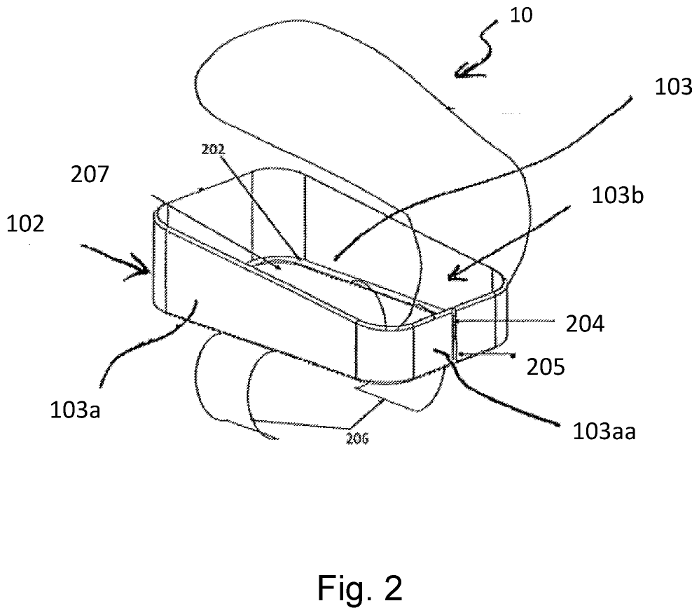

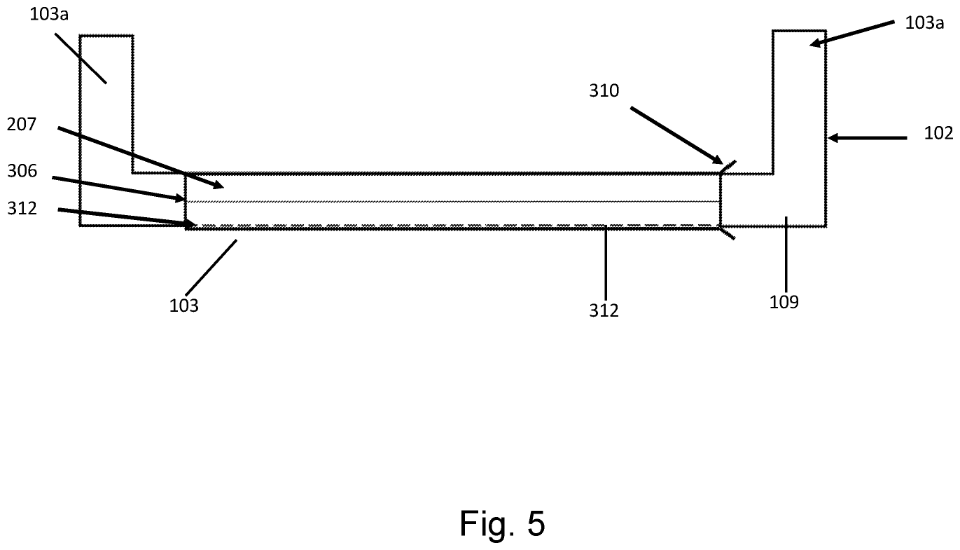

Referring to FIGS. 1 to 5, acoustic conductive cover 10 accommodates a handheld acoustic medical device in the form of an ultrasound device 101 which is in acoustic contact with the forearm 120 of a human patient. It will be understood that the acoustic medical device is not limited to ultrasound devices or ultrasound probes. Cover 10 may be applied in a range of acoustic medical procedures. Further, in other embodiments, the cover 10 may be used in veterinary procedures.

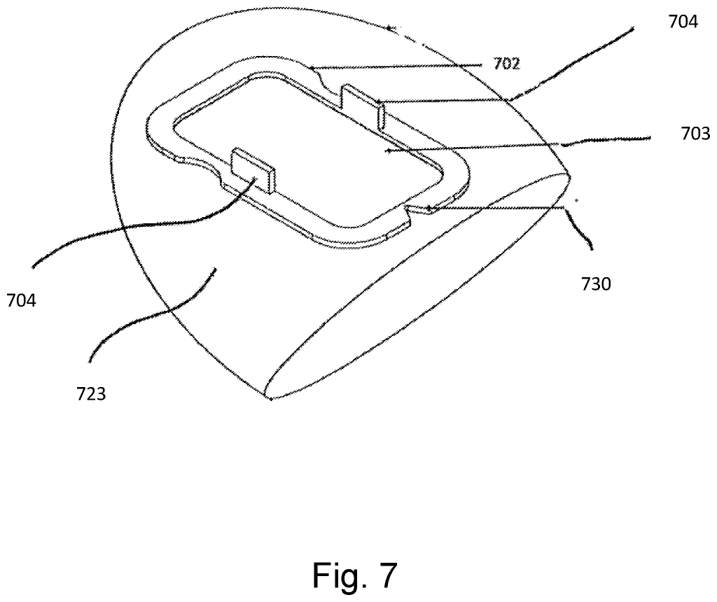

Acoustically conductive cover 10 includes, in one embodiment, a flexible frame 102. Frame 102 has a base 103 with opposed side walls 103a extending upward from the base and an open top 107. The frame 102, which in this embodiment has a generally rectangular shape, defining a cuboid volume or space 103b though other geometries suitable for housing ultrasound devices may be used. However, cuboid space 103b need not be fully enclosed by the side walls as described below with reference to FIG. 7.

Base 103 comprises an acoustically conductive membrane portion 301 as described further below and an inwardly facing lip 109 for seating the ultrasound device 101. Lip 109, as well as the side walls 103a of frame 102 do not require to be of an acoustically transmissive material.

The frame 102 may be of polymeric material and may be manufactured by a range of methods including, but not limited to: injection moulding, rotational moulding and thermocasting.

The frame 102 has sufficient material flexibility and dimensions allowing friction fitting of the ultrasound device 101 into a volume or space 103b located within it. However, in other embodiments, frame 102 may be rigid and substantially inflexible and still allow friction or interference fitting to the ultrasound device 101.

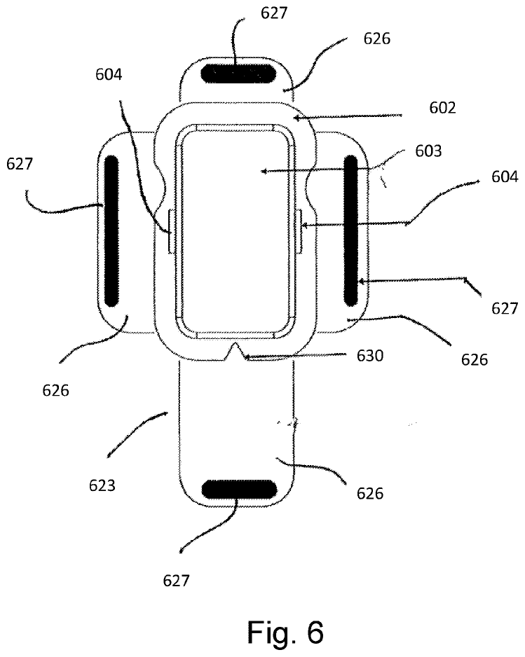

In the embodiment shown, the frame 102 accommodates the entire body of ultrasound device 101. However, in other embodiments, the frame 102 may be low profile and cover only a proportion, for example less than 90%, 50% or 30%, of the height of the side walls 103a of ultrasound device 101. The side walls 103a may not extend the full length of ultrasound device 101 as shown in FIGS. 6 and 7. The frame 102 either allows or is configured to allow viewing of a screen for ultrasound device 101, that screen preferably being on-board the device though the frame 102 or cover 10 may be provided with an in-built screen in embodiments, for example as described below.

Given the polymeric material from which frame 102 is fabricated, the frame 102, may be easily deformed to allow a tight fit to a wide range of ultrasound probes. For example, such models of ultrasound probe include those for general ultrasound such as the Samsung Accuvix XG, the Siemens ACUSON series or FUJIFILM Sonosite series probes. The models of ultrasound probe may also include hand held probes such as the GE VScan or Phillips Lumify probes. Additionally models could include acoustic devices specific for vascular access such as the Becton Dickinson Prevue II. The specific internal design of such ultrasound probes is not further described here as, with the exception of external shape already discussed, it does not affect the configuration of cover 10. In any event, such internal design of ultrasound probes is to large extent known to those skilled in the art of ultrasound probe manufacture. Similarly, use of such ultrasound probes is known to those skilled in the art of conducting ultrasound procedures.

Moreover, the ultrasound device 101 is, in preferred embodiments, as described in the Applicant's International Patent Publication No. WO2022/221913, the contents of which are hereby incorporated herein by reference. Ultrasound device 101 has a screen 104 on which data from ultrasonic imaging, for example a vein path can be displayed.

The achievable tight or friction fit between ultrasound device 101 and frame 102 allows avoidance of air gaps about the side walls. Such air gaps would cause signal noise and signal loss which may interfere with signal processing and the results provided by the ultrasound procedure and it is advantageous to avoid them.

A closure 203, in one embodiment hingeably connected to side wall 103aa of frame 102, is secured over the top of the ultrasound device 101, retaining it within space 103b. The ultrasound device 101 may not be sterile due to the need for multiple uses in a short period or difficulties in cleaning so a sterile barrier by enclosure within cover 10 is required. The closure 203, when opened, allows the ultrasound device 101 to be retained by the base 103 and side walls 103a of frame 102 forming the cover 10.

The closure 203 is conveniently formed from an acoustically conductive polymer film which may be of cling film or provided with adhesive, for example disposed in the form of adhesive strips along edges 203a of closure 203. The polymer film is desirably transparent as this allows easier operation of ultrasound device 101.

When the closure 203 is closed by adhesion to the side walls 103a of frame 102, the cover 10 is closed and this enclosure of the ultrasound device 101 with a barrier between space 103b and the sterile area at the patient's forearm 120 provides protection against infection. The cover 10 enables the ultrasound probe 101 to be installed in the cover 10 by a single user which tackles a major clinical problem of prior sheathing in ultrasound guided procedures which requires two operators as described above.

Referring to FIGS. 1 to 3, fixation straps 106, for example including Velcro fasteners, are connected to frame 102 to allow connection of the covered ultrasound device 101 to the patient's forearm 103 whilst allowing for hands-free use of the ultrasound device 101 in a sterile manner, leaving the user's hands free for performing a medical procedure such as a cannulation. Other fixation means could be used including, without limitation: sterile medical grade adhesive liners and mechanical fasteners. The fixation straps 106, or other connection means, may be adapted for connection to other parts of the body, for example, legs, torso or abdomen where the length of the fixation straps can be made longer. The fixation straps 106 may be loosened or tightened as necessary and may act as a tourniquet.

In embodiments, the procedure being undergone by the patient may be a venipuncture or cannulation involving a vascular catheter. Guiding the catheter to a suitable vein is greatly assisted by real time ultrasound imaging as described in the Applicant's International Publication WO2022/221913 incorporated herein by reference. The inclusion of a needle alignment guide 204 and needle guide 205 provides guidance to the health professional using ultrasound device 101 on catheter insertion during the venipuncture or cannulation procedure.

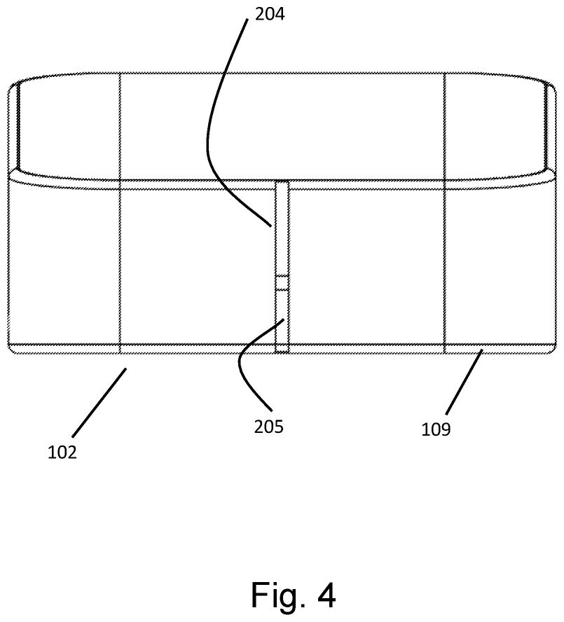

As shown in FIG. 4, the needle alignment guide 204 cuts through the middle of side wall 103aa of the frame 102 to align the needle guide 204 with the top of the screen 104 of ultrasound device 101, preferably as described in the Applicant's International Publication WO2022/221913 incorporated herein by reference. Needle guide 205 is a sloping triangular shaped notch or indent in side wall 103aa extending to the base 103 of frame 102.

In embodiments, frame 102 includes a receptacle in the form of patch or pocket 207 for containing an acoustic coupling medium in the form of a conventional ultrasound gel as known in the art of conducting ultrasound procedures. Pocket 207 is continuous with base 103 of frame 102. This pocket 207, together with the acoustically conductive base 103, provides direct conduction of acoustic waves from the ultrasound device 101 to the imaging area on the patient's forearm 120 in a sterile manner.

The pocket 207 has volume to retain a determined volume of gel for the ultrasound procedure, this avoiding the need for use of additional ultrasound gel, simplifying the workflow of an ultrasound procedure. Such volume of ultrasound gel is most preferably less than 90 cm³ , for example 50 cm³ and may be 30 cm³ or less.

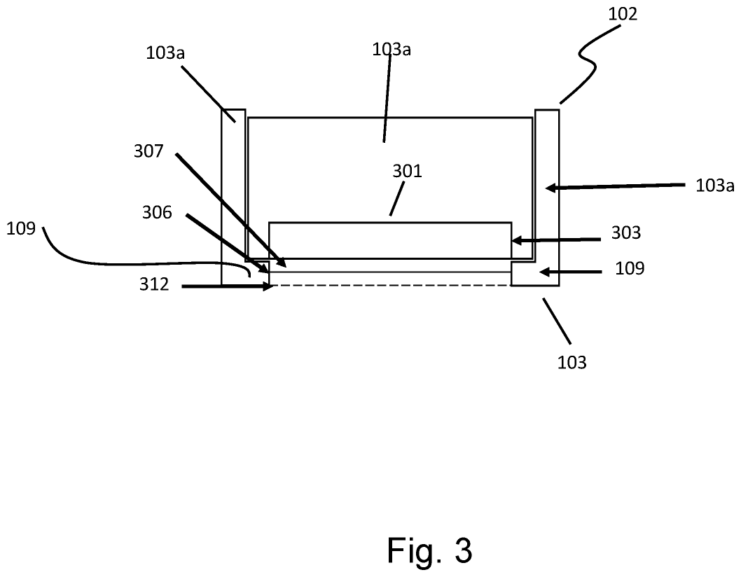

Referring to FIG. 3, the ultrasound transducers in transducer array 303 contact the ultrasound gel in the pocket 207. In further detail, the pocket 207 comprises an acoustically transmissive membrane portion 301 comprising a perforated membrane 306 and an unperforated membrane 307 disposed in parallel, the unperforated membrane 307 blocking contact with the patient's forearm to maintain a sterile imaging area. This enables direct coupling of transducers in the transducer array 303 to the imaging area. It will be appreciated that perforated membrane 306 and unperforated membrane 307 form, with the gel, the acoustically conductive portion 301 of the base 103.

Perforated membrane 306 is a thin ultrasound transmissive polymer film with small gaps or pores, i.e. perforations, to allow passage of ultrasound gel. This allows for ultrasound gel to pass through the perforations upon compression of cover 10 against the imaging area of interest on the patient's forearm 120. This removes the need for adding extra ultrasound gel between the cover 10 and the imaging area, simplifying the workflow of the ultrasound procedure.

Unperforated membrane 307 is a thin ultrasound conductive polymer film that allows acoustic conductance of ultrasound, whilst maintaining a physical sterile barrier between the ultrasound device 101 and the imaging area to maintain sterility.

Above perforated membrane 306 is located an acoustic gel peel-off upper liner 310 which is a thin plastic film removable to expose sterile ultrasound gel as filled into pocket 207. This liner is peeled off before seating the ultrasound device 101 on the base 103 of the frame 102.

Below perforated membrane 306 is located an acoustic gel peel-off lower liner 312 which is removed to expose the perforated membrane 306 when the ultrasound device 101 is retained by frame 102.

FIG. 6 shows a further embodiment of frame 602 having a base 603 the same in construction as base 103 described above but which does not have side walls extending around all sides 602a. Rather, the side walls 604 are provided as two shorter walls or alignment guides extending from two sides of the base 603 at about the mid point of two sides of the base 603 of the frame 602. It will be understood that other arrangements of base 603 and alignment guides 604 are possible. For example, multiple side walls or alignment guides 604 could be provided on each side of the base 603 of frame 604. Such alignment guides could also be provided at each end of the base 603 of frame 604. However, the arrangement shown allows friction fitting of a hand held ultrasound device 101 between alignment guides 604 and avoids air gaps which may interfere with ultrasound signal transmission. As shown in FIG. 6, the acoustically conductive closure 623 is again a transparent film with four flaps 626 that ease wrapping and enclosure of frame 602. The flaps 626 have adhesive strips 627 allowing adhesion to adjacent flaps 626 to enclose the ultrasound device 101. Ultrasound gel is accommodated in pocket 207 which is the same as described above. Frame 602 is also provided with a needle guide in the form of a triangular notch 630 to guide needle insertion as described above.

FIG. 7 shows a third embodiment of frame 702 having a base 703 the same in construction as bases 103 and 603 described above but which do not have side walls extending around all sides 702a of frame 702. Rather, the side walls 604 are provided as shorter walls or alignment guides 704 extending from two opposed sides of the base 703, in the embodiment shown at about the mid point of two sides of the base 703 of the frame 702. As with the frame 602, other arrangements of alignment guides 704 are possible provided that hand held ultrasound device 101 can be fitted, desirably friction fitted for avoidance of air gaps, between them. Frame 702 is also provided with a needle guide in the form of a triangular notch 730 to guide needle insertion as described above.

As shown in FIG. 7, the acoustically conductive closure 723 surrounding the frame 702 is again an acoustically conductive transparent film but, in this embodiment, formed as a sleeve or sheath which is connected to, and encloses, the frame 702 and ultrasound device (not shown but in the same form as ultrasound device 101). By fitting the unsterilised ultrasound device into frame 702 and placing a sterile-gloved hand into the opening 723a of the polymer sheath 723, the ultrasound device can be covered in a sterile manner by one user through, for example, turning the sheath 723 in on itself and over the ultrasound device. The closure may be made more secure through closing the opening with the use of an elastic band or, more preferably, an adhesive lining that allows detachable fixing of the sheath 723 to the ultrasound device.

Cover 10 may be re-usable on sterilisation or disposable.

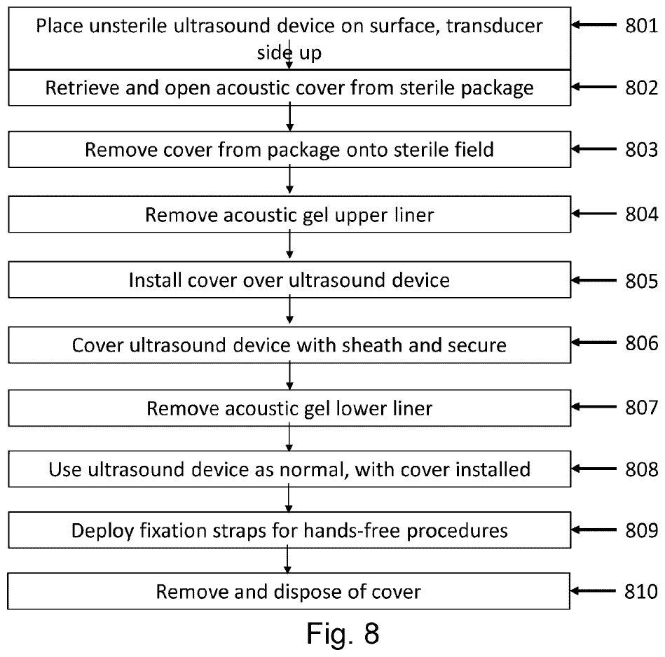

Referring to FIG. 8, there is shown an exemplary workflow for using the cover 10 as described above. The workflow is made up of a series of steps that can be completed by a single operator or user of ultrasound device 101.

First, step 801, the unsterile ultrasound device 101 is placed on a work surface with the base 103 and transducer array 303 facing upwards.

Second, step 802, the packaged sterile consumable acoustic conductive cover 10 is retrieved and the package is opened.

Third, step 803, the cover 10 is removed from the package into a sterile field proximate a patient.

Steps 801 to 803 can be completed by the user whilst they are unsterile, provided that they are only touching the packaging and not the sterile field or cover 10.

Once the user is sterile, i.e. has applied sterile gloves using sterile techniques as known in the art, remaining steps 804 to 810 can be conducted.

Fourth, step 804, the acoustic gel peel-off upper liner 310 is removed to expose sterile ultrasound gel in the membrane portion 301 of base 103 of cover 10 prior to seating of the ultrasound device 101 on lip 19 of the base 103.

Fifth, step 805, cover 10 is deployed to locate the ultrasound device 101 such that cover 10 touches the transducers of transducer array 303.

Sixth, step 806, closure 203 is positioned to close the space 103b defined by the base 103 and side walls 103a of cover 10. At the end of step 806, ultrasound device 101 is completely enclosed by cover 10 and a sterile barrier between patient and ultrasound device 101 is available.

Seventh, step 807, the acoustic gel peel-off lower liner 312 is removed to expose the perforated acoustic membrane 206 allowing expression of ultrasound gel on compression of ultrasound device 101 and cover 10.

Eighth, step 808, the covered ultrasound device 101 can be used in a sterile manner as normal as it can still be operated by the user when covered by cover 10. Ultrasound gel can be delivered through perforated acoustically conductive membrane 206 to the imaging area on the patient's forearm 150 on compression of the covered ultrasound device 101 against the imaging area. This enables acoustic conductance from the transducer array 303 to the imaging area of interest.

Ninth, step 809, fixation straps 106 can be deployed, as shown in FIG. 1, to connect the covered ultrasound device 101 to the patient's forearm 150. This allows the user to avoid holding the covered ultrasound device 101 in position on the patient's forearm during the procedure, for example cannulation or venipuncture. The needle guides 204 and 205, together with ultrasound guiding of the catheter using ultrasound device 101 also assist efficient performance of the procedure.

Tenth, step 810, once ultrasound imaging is completed, cover 10 is removed from ultrasound device 101 and disposed of or, in some embodiments, sterilised for re-use.

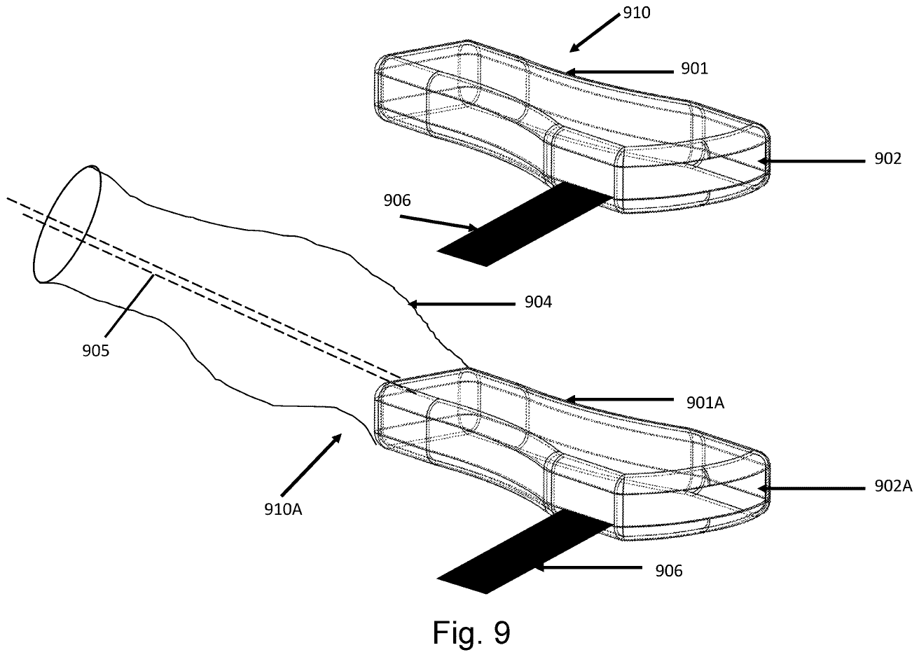

FIGS. 9(a) and 9(b) show how a sterile consumable acoustic conductive cover may be used to cover both wireless and conventional wired ultrasound probes 901 and 901A.

As shown in FIG. 9(a), cover 910 includes a rigid polymer frame 902 which is configured in the shape of, and surrounds, wireless ultrasound probe 901.

As shown in FIG. 9(b), cover 910A includes a rigid polymer frame 902A is configured in the shape of, and surrounds, conventional wired ultrasound probe 901A. Additionally, a cylindrical tube 904 of polymer film is connected to the frame 902A. Tube 904 can be pulled over the probe wire 905, protecting the user-such as a health professional-where the probe wire 905 is unsterile.

Both covers 910 and 910A conveniently comprise fixation straps 906, the same in form as described for fixation straps 106 above.

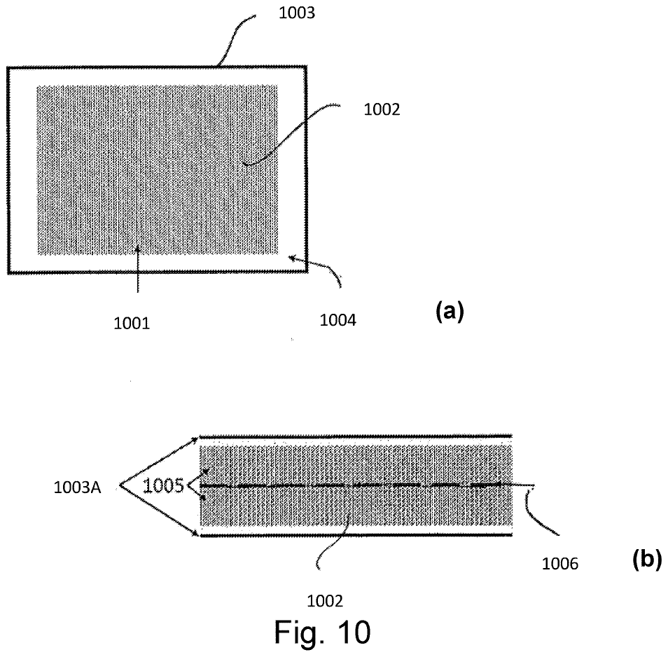

FIGS. 10(a) and 10(b) show, without limitation of other options, different configurations of gel receptacle for use in an ultrasound procedure analogously to pocket 207.

FIG. 10(a) shows a gel receptacle 1001 of one embodiment manufactured using a form fill seal method to encase gel 1002 in a pocket or sheath 1003 of polymer material. A layer of sheath border 1004 is stamped around the gel receptacle 1001 to allow assembly with a frame similar to frames 102 and 902 described above.

FIG. 10(b) shows a gel receptacle 1001A in sectional view. Gel receptacle 1001A comprises a solid sterile barrier 1006 surrounded by gel 1005 that is suspended by the solid sterile barrier 1006. Two peelable layers 1003A surround the gel to preserve sterility of the gel before use. Once the peelable layers 1003A are removed the gel, in adjacency with transducer array 303, is exposed for use in an ultrasound procedure.

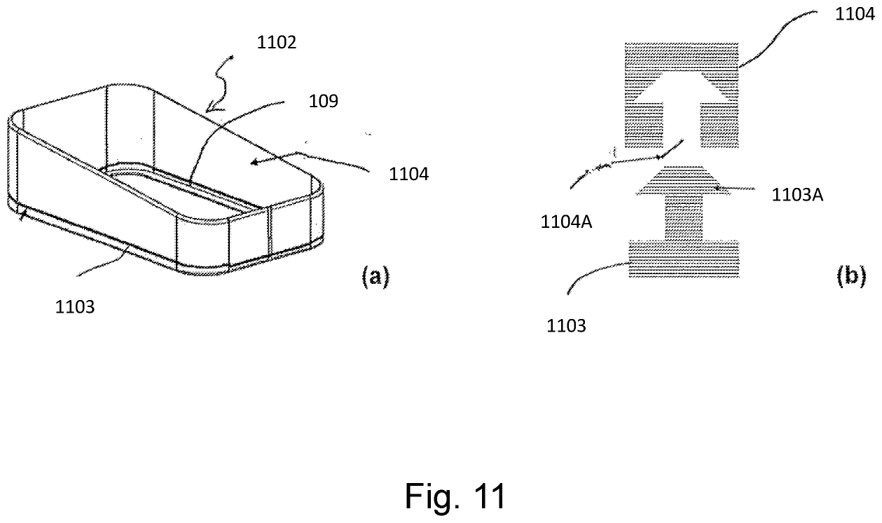

FIG. 11(a) shows a further embodiment of frame 1102 for use in cover 10. Frame 1102 comprises a lower or base portion 1103 and an upper portion 1104 manufactured using injection moulding. The base and upper portions 1103 and 1104 may be press-fitted together. This is shown schematically in the exploded section view, FIG. 11(b), where a protrusions 1103A can fit within upper slots 1104A in a tongue and groove connection. It will be understood that different shapes of protrusion 1103A can be used. Further, different forms of connection between base and upper portions 1103 and 1104 may be employed.

Other form factors for the acoustic cover are possible and exemplary embodiments are described further below.

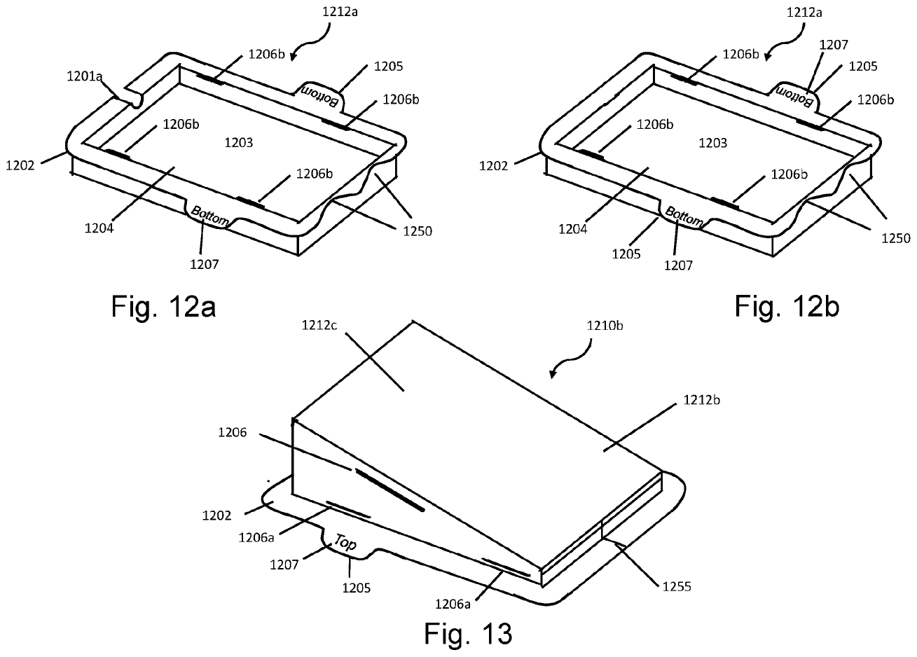

Referring to FIGS. 12 to 15, the acoustic cover 1210 comprises a two part frame 1212 having a combination of two components: a bottom frame portion 1212a and a top frame portion 1212b which may be separate or hingedly connected. Frame 1212 resembles a strawberry punnet. Frame portions 1212a and 1212b combine to fully enclose an acoustic medical device, such as ultrasound device 101, tightly fitted within a cavity including cavity 1204 of bottom frame portion 1212a. Such fitting together of frame portions 1212a and 1212b is achieved using complementary protrusions and grooves 1206a and 1206b. Ultrasound device 101, when properly fitted has its screen proximate the upper optically transparent surface 1212c of cover 1210 and its transducer arrays seated proximate optically and acoustically transparent membrane 1203.

In this manner, a sterile barrier is provided between the unsterile ultrasound device 101 and a patient throughout a range of medical and veterinary procedures. It will be understood that the acoustic medical device enclosed by acoustic cover 1210 is not limited to ultrasonic devices or ultrasonic probes.

Referring to FIGS. 12(a) and 12(b), these show two separate embodiments for the bottom frame portion 1210a. FIG. 12(a) shows that bottom frame portion 1212a is compatible with a wired acoustic device through provision of a cable hole 1201a. FIG. 12(b) shows a bottom frame portion 1212a that would be compatible with a battery-operated acoustic device as it has no cable hole 1201a.

Bottom frame portion 1212a is rigid and, in this embodiment, has a generally rectangular shape. It will be appreciated that bottom frame portion 1212a and frames described herein do not require a rectangular shape as a range of form factors may be apparent to the skilled reader of this disclosure. For example, frames or frame portions could—subject to ergonomic comfort of a user—be circular, oblong or other shapes.

Acoustic medical device 101, for example, would be accommodated within the internal volume 1203 of the acoustic cover 1210.

Bottom frame portion 1212a includes an acoustically transparent membrane 1204 which may conveniently be provided with a predetermined volume of ultrasonic or other acoustic couplant to be used throughout the required procedure, whether medical or veterinary.

Bottom frame portion 1212a may also be provided with means for improving grip of the acoustic cover 1210 by a user. One option, as shown in FIGS. 12(a), (b) and 13, is to provide, both the bottom frame portion 1212a and top frame portion 1212b with an overhanging brim 1202 and grooves 1250 providing gripping points. There are also two tabs 1205 which have indented text annotations 1207 of ‘Top’ or ‘Bottom’. As shown, ergonomically comfortable thumb grooves are also provided to improve the user's grip.

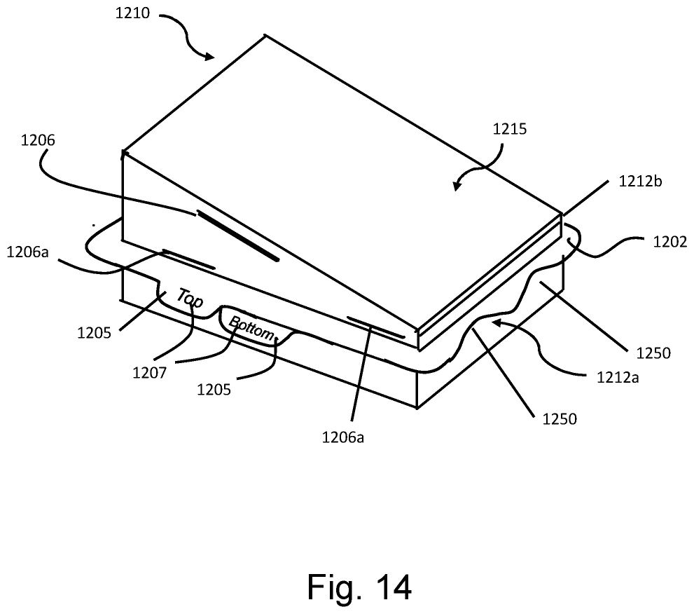

FIG. 14 shows the two part cover 1210 formed from fitting together bottom frame portion 1212a and top frame portion 1212b which has a wedge shape with an inclined upper surface and is also rigid. Top frame portion 1212b includes similar grip improving features as described in relation to bottom frame portion 1212a. Thus, top frame portion 1212b includes overhanging brim 1202, two tabs 1205 with indented text annotations 1207 and grooves 1206a to fit complementary protrusions 1206b of the bottom frame portion 1212a.

Top frame portion 1212b also includes an optically transparent portion 1215 which is intended to sit on a screen of the acoustic device, for example, screen 104 of ultrasound device 101. Marking 1255 is included to indicate the centre of the ultrasound device 101. Internal indentations 1206a are provided to allow secure fixing of the acoustic medical device 101 to the bottom frame portion 1212a by engagement with complementary protrusions 1206b.

FIG. 14 shows the acoustic cover 1210 formed from the combination of a bottom frame portion 1212a and top frame portion 1212b. When fitted together, these components completely enclose or encapsulate the acoustic medical device (with no movement of device within cover 1210) while still permitting ultrasonic medical procedures to be performed.

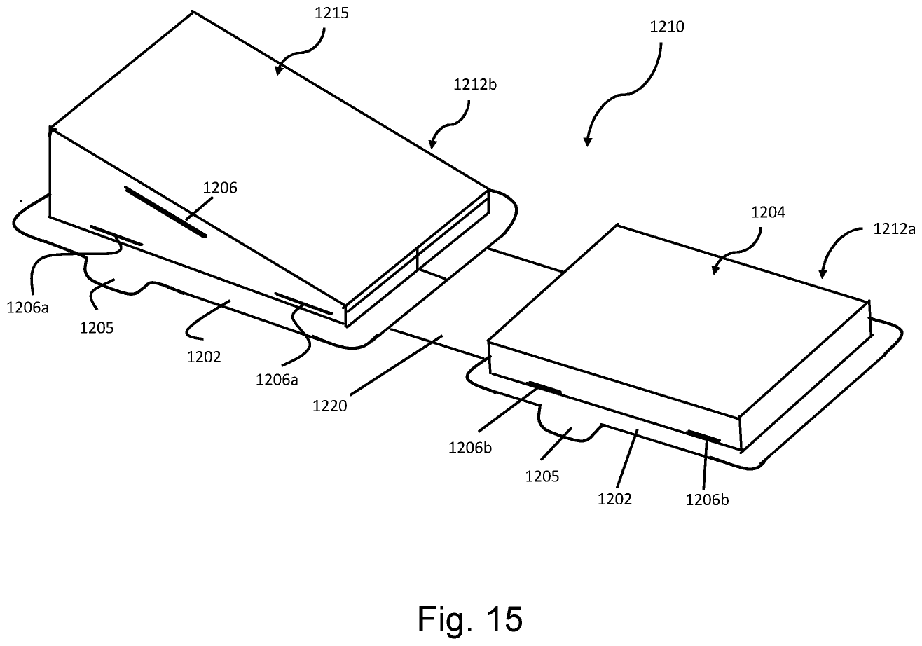

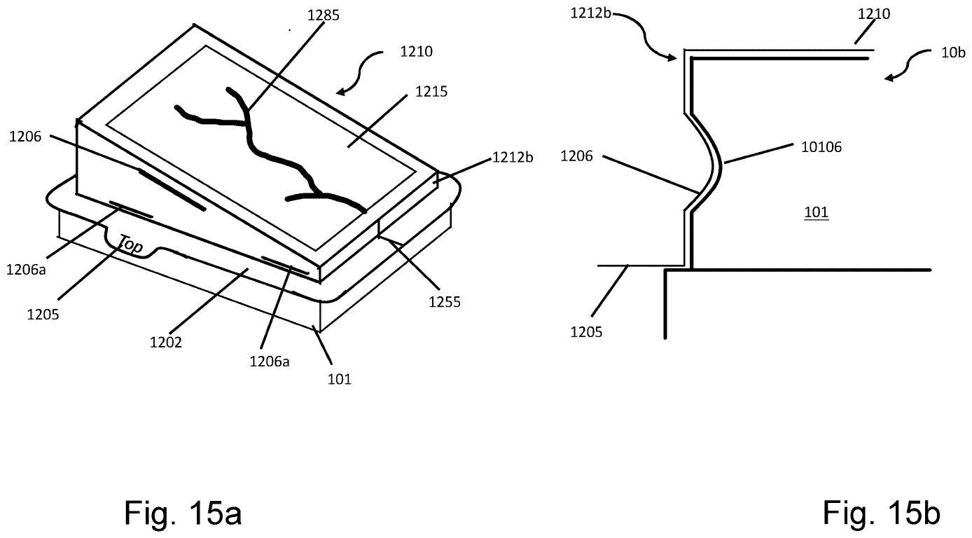

FIG. 15 demonstrates the potential for a similar embodiment in which bottom frame portion 1212a and top frame portion 1212b, with structure as described above, are joined by a thin flexible bridge 1220. Flexible bridge 1220, which may act as a hinge, may be made of a polymeric material. The material, as with the material used to fabricate bottom frame portion 1212a and top frame portion 1212b, may be recyclable.

FIG. 15A shows the top frame portion 1212b of cover 1210 fitted to an acoustic medical device, here a portable ultrasound device 101. When connected, by engagement of bottom grooves or indentations 1206a, with complementary protrusions 1206b in the inside of the bottom frame portion 1212a (conveniently by a snap fit), ultrasound device 101 is tightly fitted within the cover 10 allowing convenient use of the probe. FIG. 15B shows the connection between ultrasound device 101 and cover 1210 where upper protrusion or indentation 1206, elongated in comparison with bottom grooves or indentations 1206a, engages with a corresponding groove 10106 of ultrasound device 101. Indentation 1206 and groove 10106 allow alignment of the ultrasound device 101 within cover 1210 and a secure connection. In the embodiments shown, there are multiple indentations 1206 and corresponding grooves on the ultrasound device 101. These indentations 1206 and corresponding grooves may be spaced as convenient about the cover frame portions 1212a and 1212b as well as the ultrasound device 101. Locations of indentations and corresponding grooves may be reversed and different connection arrangements may be adopted, including within the same cover 1210 and ultrasound device 101.

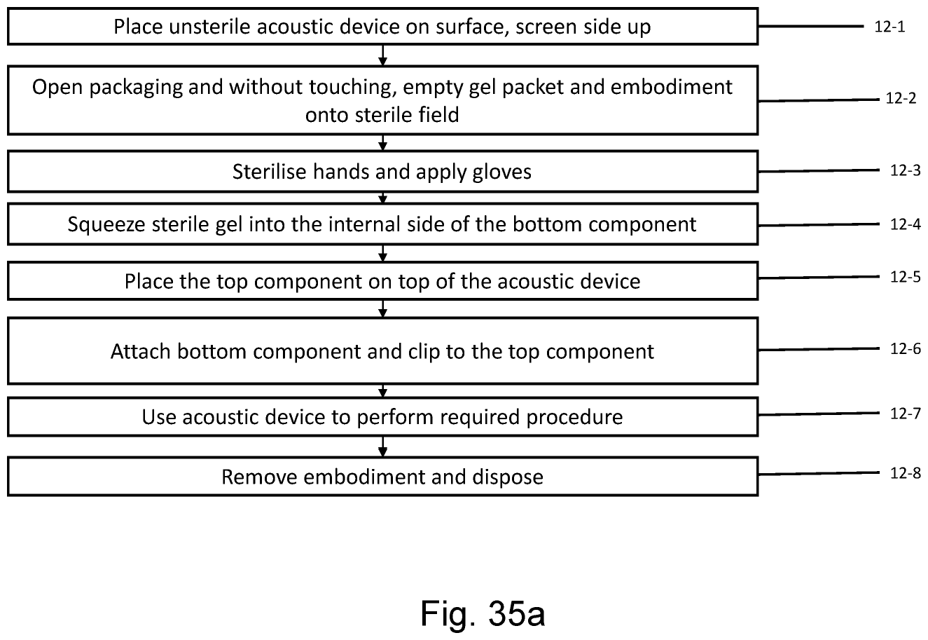

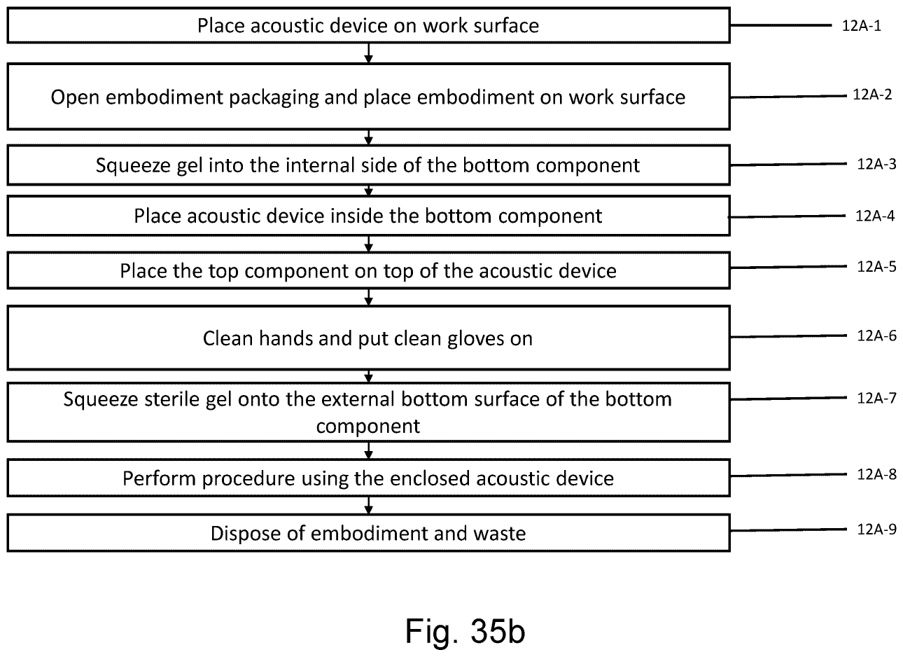

Referring to FIG. 35(a), there is shown an exemplary workflow for a sterile procedure using a two part cover such as cover 1210 as shown in FIGS. 12 to 15B in which bottom frame portion 1212a and top frame portion 1212b are attached, conveniently by being snap fitted or clipped together with the frame portions 1212a and 1212b including the protrusions 1206b having sufficient elasticity to permit this. It will be understood that alternative workflows may be followed though the principle of maintaining a sterile barrier between patient and ultrasound device 101 will be maintained where sterile, as opposed to clean, procedures are to be conducted.

In step 12-1, the unsterile ultrasound device 101 is placed on a non-sterile surface, such as a table top, screen side up.

In step 12-2, the sterile, packaged cover 1210 is opened by removing the packaging. Packaging for cover 1210 may be conventional medical packaging using either a polymer package or a box. Without touching the cover 1210, it is placed on a sterile field.

In step 12-3, the user sterilises their hands and gloves are worn.

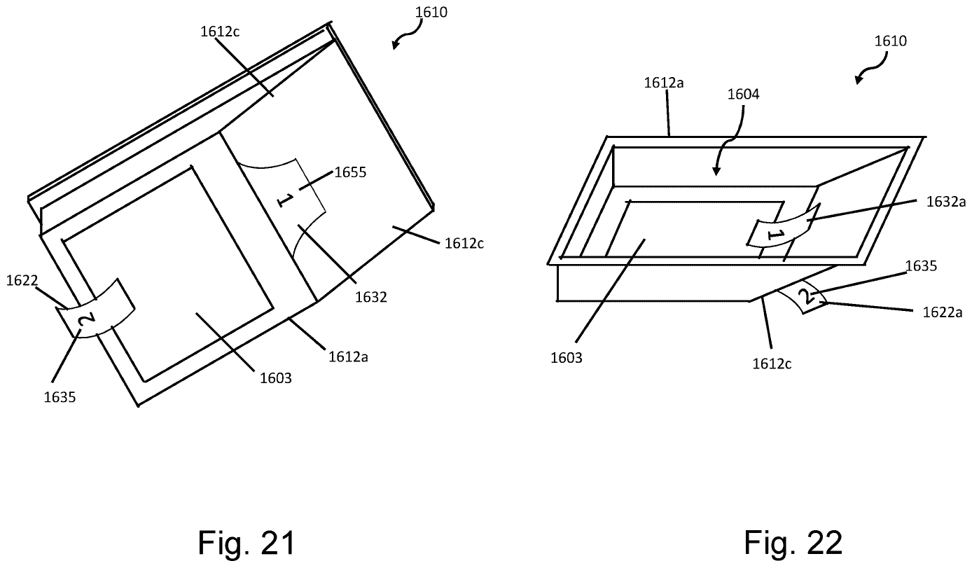

In step 12-4, a quantity of acoustic couplant is applied to the internal surface 1204 of the bottom frame portion 1212a for location proximate the transducer array. Conveniently, the source of acoustic couplant is a sachet containing it. Alternatively, the gel may be already contained in the bottom frame portion 1212a and exposed as described with reference to FIGS. 21-24.

In step 12-5, the top frame portion 1212b is placed onto the screen of the ultrasound device 101 with the ultrasound device 101 fitted within it.

In step 12-6, the ultrasound device 101 is lifted by gripping the top frame portion 1212b and the bottom frame portion 1212a is then attached to the top frame portion 1212b and clipped or snap fitted into position (using engagement between protrusions 1206b and grooves 1206a) such that the two frame portions 1212a and 1212b form the cover enclosing ultrasound device 101.

In step 12-7, the cover 1210 is sterile on all external surfaces and can be picked up by a user to perform a procedure using the enclosed ultrasound device 101. The procedure may include adding an acoustic couplant to the patient through a provided sachet or included in the base of the cover 1210 itself, as described with reference to FIGS. 21-24. During the procedure, the user takes advantage of the ergonomically efficient cover design, as described above, to allow more precise movement of the cover 1210 and conduct the procedure, such as cannulation, more safely and efficiently.

In step 12-8, following conduct of the procedure, the cover 1210 can be removed and disposed of or recycled. The ultrasound device 101 is reusable when provided with a further sterile cover such as cover 1210.

Referring to FIG. 35(b) there is shown an exemplary workflow for a clean procedure using the two part cover such as cover 1210 shown in FIGS. 12 to 15B. It will be understood that alternative workflows for a clean, as opposed to a sterile procedure, may be followed.

In step 12A-1, the acoustic device 101 is placed on a table, bench or otherwise nearby work surface to be used during the clean medical or veterinary procedure.

In step 12A-2, the sterile, packaged cover 1210 is opened by removing the packaging. Packaging for cover 1210 may be conventional medical or veterinary packaging using either a polymer package or a box. Cover 1210 is placed on the same work surface as acoustic device 101.

In step 12A-3, a quantity of acoustic couplant is applied to the internal surface 1204 of the bottom frame portion 1212a. Conveniently, the source of acoustic couplant is a sachet containing it. Alternatively, the gel may be already contained in the bottom frame portion 1212a and exposed as described with reference to FIGS. 21-24.

In step 12A-4, the acoustic device 101 is placed into the bottom frame portion 1212a so that the transducer array is in contact with the acoustic couplant gel.

In step 12A-5, the top frame portion 1212b is fitted to the bottom frame portion 1212a enclosing the acoustic device 101 with a frictional grip.

In step 12A-6, the user cleans hands and puts clean gloves on.

In step 12A-7, sterile acoustic gel is applied to the external surface of the base of the bottom frame portion 1212a.

In step 12A-8, the procedure is performed using the covered acoustic device 101 with the user taking advantage of the ergonomically efficient cover design, as described above, to allow more precise movement of the cover 1210 and conduct the procedure, such as cannulation, more safely and efficiently.

In step 12A-9, following conduct of the procedure, the cover 1210 can be removed and disposed of or recycled. The ultrasound device 101 is reusable when provided with a further cover 1210.

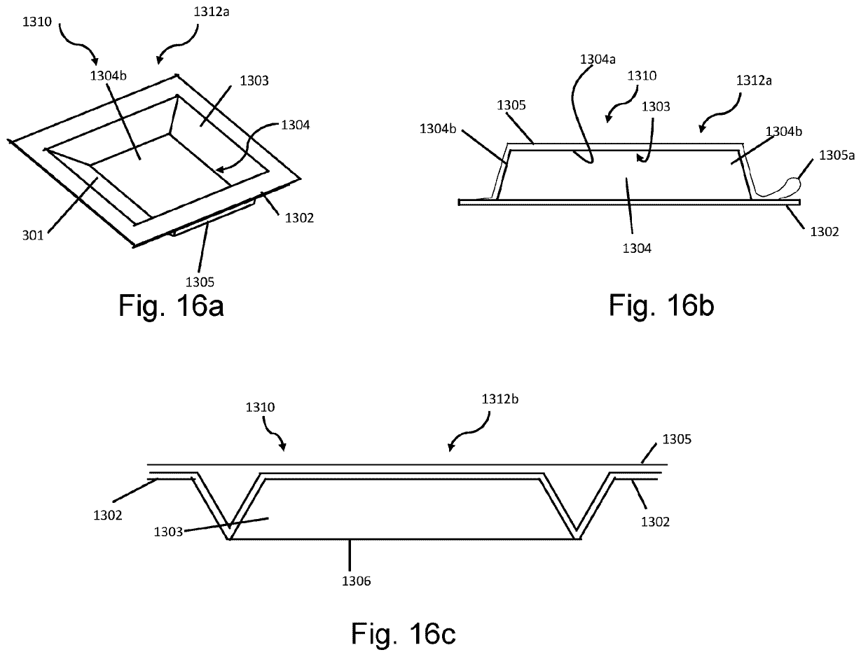

In another embodiment as shown in FIG. 16(a) to (c), the cover may include an adhesive peel layer on outer surface(s) in locations that are likely to be in contact with patients. Advantageously, this embodiment would maintain the sterility of those key areas and allow for the frame to be transported and distributed without the need for sealed secondary packaging for transport. This embodiment allows reduction of the materials required to manufacture the frame as well as the amount of materials going to waste after the device has been opened and used. In following figures, features have the same numbering except for a prefix of the figure number, e.g. groove 1906a, unless otherwise indicated.

As with the embodiment of FIGS. 12 to 15B, the frame 1310 of FIG. 16(a) to (c) comprises a bottom frame portion 1312a and a top frame portion 1312b which are fitted together to enclose the acoustic medical device, for example ultrasound device 101 which can sit against concave surface 1304b of acoustically transparent membrane 1303.

Concave surface 1304b is non-sterile but clean whereas, on the convex side 1304, a thin membrane 1305 is able to be peeled off at a tab 1305a, exposing a sterile surface 1304a which can be used during the required procedure to maintain sterility.

FIG. 16(a) shows the non sterile cavity 1304 of the bottom frame portion 1312a which is able to house the acoustic medical device, for example ultrasound device 101. Bottom frame portion 1312a of frame 1310 is rigid and configured to fit the acoustic medical device via a friction fit or similar method. There is also an overhanging brim 1302 on an external surface of bottom frame portion 1312a to improve grip and usability. On the bottom surface of bottom frame portion 1312a is provided an ultrasonically transparent membrane 1304. Provision may also be made for storing or adding a predetermined amount of sterile ultrasonic couplant.

FIG. 16(b) shows the underside of the cavity 1304 of the bottom frame portion 1312a. The underside of cavity includes a thin peelable layer 1305. Once peeled, this layer exposes an external sterile and acoustically transparent surface 1304a to contact a patient's skin during the medical procedure, for example cannulation. The thin peelable layer 1305 may be made of a suitable medically applicable material, for example a breathable material such as the non-woven material produced by Dupont under the trade mark Tyvek. As another example, a polyethylene, preferably LDPE, could also be used.

FIG. 16(c) shows the upper frame portion 1312b which is provided with a thin peelable layer 1305a of the same kind as described for bottom frame portion 1312a. Top frame portion 1312b is also rigid and is also provided with an overhanging brim 1302a to improve grip and usability. Also included is an optically transparent barrier 1306 to enable the screen of the acoustic device to be seen even when enclosed.

Frame portions forming the cover may be fitted or locked together in various ways to ensure that the acoustic device is enclosed during a medical or veterinary procedure. Without limitation, fitting together of the frame portions may include friction, adhesive and snap fit joints. Advantageously, such fits may also include means to allow guidance of finger or thumb placement while operating the acoustic device.

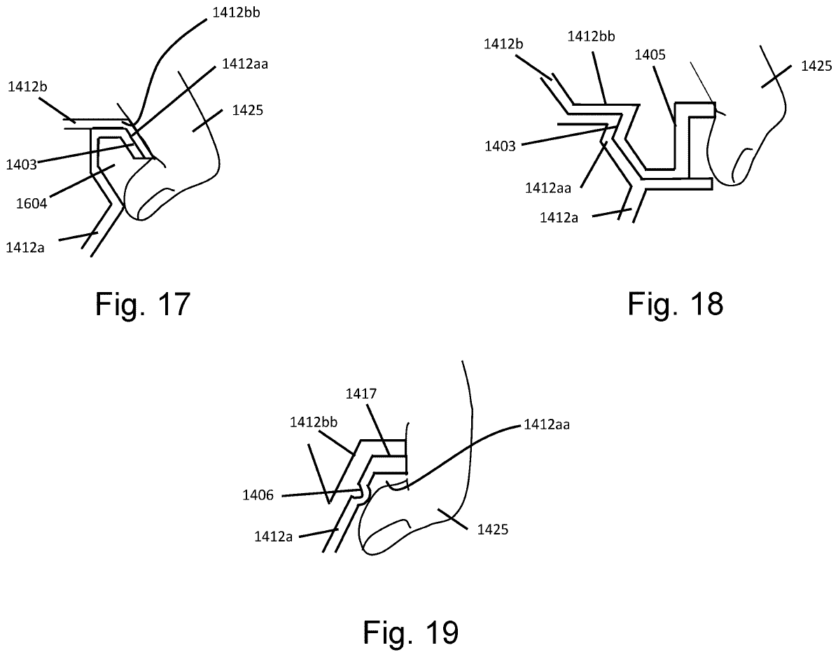

FIGS. 17 to 19 show a number of embodiments applicable for fitting together of top and bottom frame portions of a two part cover, such as those described above. The locking means may also serve as a grip point where a user's finger(s) can sit comfortably during use.

FIG. 17 shows an external locking means 1403 between the top frame portion 1412b and bottom frame portion 1412a. Provision of a cavity 1404, in the form of a channel having a V shaped base, allows accommodation of a user's finger or thumb 1425, providing a comfortable grip. It will be understood that cavity 1404 need not have a V shaped base and could have another ergonomically effective geometry.

FIG. 18 shows a locking means 1403a to FIG. 17. However, locking means 1403a also includes an extended top component tab 1405 which provides an alternative form of cavity enabling comfortable finger gripping and handling during use and, consequentially, a safer and more efficient procedure.

FIG. 19 shows how an internal locking means 1406 can be achieved by the bottom frame portion 1412aa encompassing the top frame portion 1412bb. The internal locking means 1406 may optionally include a small indentation 1407 to acts as a locking means between frame portions 1412aa and 1412bb. When the frame portions 1412aa and 1412bb are fitted together, a small overhanging tab 1417 is formed providing a grip point for a user's fingers during use.

In embodiments, the cover is desirably configured to allow it to be comfortable hold without impeding the functionality of either the cover or the acoustic device which it encloses. Such configuration which may be applied to any of the embodiments of cover or frame portions described above may include portions for facilitating user grip and making the grip more comfortable. Such portions may preferably, though without limitation, include thumb grooves, curved edges, grip points together with ergonomically effective sizing of such portions. By ‘ergonomically effective’ is intended that most users find the cover easy to hold and comfortable to use.

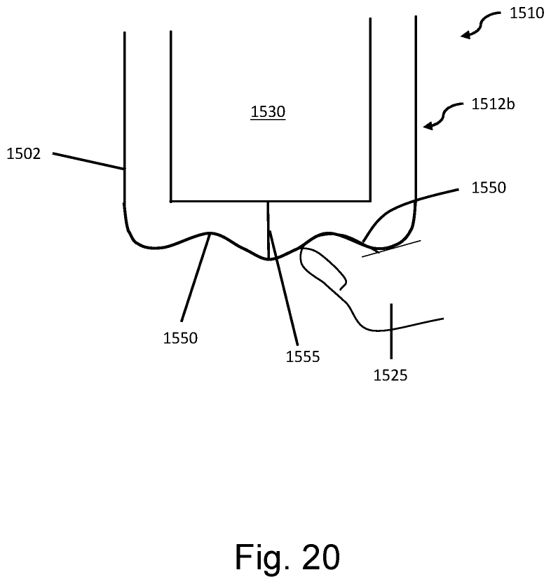

FIG. 20 shows the top frame portion 1512b of a two part cover 1510 with an ergonomically effective configuration. Cover 1510 is provided at one end with two grooves or indentations 1550, here arranged in parallel, in an overhanging brim 1502 to allow for comfortable finger or thumb 1525 placement during use. The location of these grooves or indentations 1550 is—provided ergonomically effective—optional and they may be provided at ends, sides, top or bottom or any combination of these. Preferably, the location(s) of the groove(s) or indentation(s) are located so as not to impair viewing of the central marking 1555 of the top frame portion 1512b or screen as visible through optically transparent membrane 1530. Alternatively or additionally, such groove(s) or indentation(s) 1550 may be provided on a bottom frame portion of two part cover 1510 as described for the embodiments above.

Such grooves or indentations 1550 are also located to prevent hand, finger or thumb 1525 contact with areas of operational importance from the perspectives of either maintaining sterility, preventing obstruction of energy beams and so on.

Embodiments of the cover as described herein advantageously include peelable layers, which once peeled, expose acoustic couplant to permit the use of acoustic devices throughout a required medical or veterinary procedure. Such peelable layers may include indicia or other features facilitating use of the covers described herein. FIGS. 21 to 24 show such embodiments including peelable tabs present on internal, external or both surfaces of the frame portions forming a cover in some embodiments. A peelable layer may be adhered with adhesive or heat welded to such surfaces.

FIG. 21 shows an embodiment in which a wedge shaped rigid bottom frame portion 1612a of a two part cover 1610 for accommodating a wedge shaped acoustic medical device is provided both with an external tab 1622 and an internal tab 1632. External tab 1622 is peeled from one end of sloping wall 1612c. Tabs 1622 and 1632 are attached to a thin membrane which may be easily peeled to expose underlying ultrasonic couplant 1640 which will be located proximate the transducer array of an acoustic medical device providing a better signal to noise ratio and higher resolution imaging. Tabs 1622 and 1632 may include indicia such as numbers or other text indicators 1635 to describe in which order the peelable layers should be peeled off or removed, e.g. ‘1’ meaning first and ‘2’ meaning second. Such indicia could be replaced by letters, e.g. ‘A’, ‘B’ or colours, e.g. ‘Red', 'Green’ and many other options are available. Such indicia may also be used in conjunction with instructions for use of the cover which may be provided for any of the embodiments of cover described herein.

FIG. 22 shows an alternative embodiment to FIG. 21 in which the internal tab 1632a is peelable from the inside of the cavity 1603 defined by bottom frame portion 1612a. The external tab 1622a is peeled from the external surface of bottom frame portion 1612a at the bottom of sloping wall 1612c.

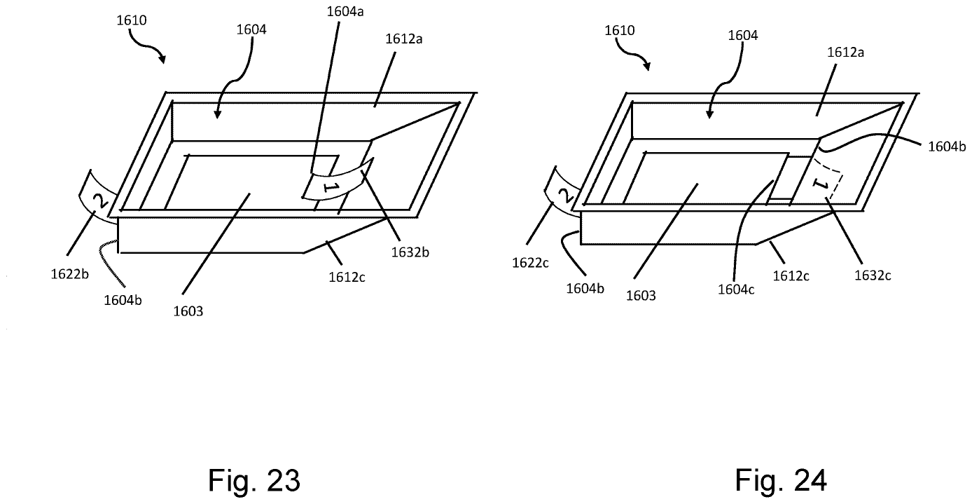

FIG. 23 shows an alternative embodiment to FIGS. 21 and 22 in which the internal tab 1632b is peelable from the inside wall 1604a of the cavity 1604 defined by frame portion 1612. The external tab 1622b is peeled from an external surface 1604b on the opposite side of the inside wall 1604a of bottom frame portion 1612a.

FIG. 24 shows a further embodiment to FIGS. 21 to 23 in which the internal tab 1632c is peelable through a slot 1604c from the external surface 1604b of the cavity 1604 defined by frame portion 1612. The external tab 1622c is also able to be peeled from the external surface 1604b.

The cover may be provided with functionality, for example a power source for powering the acoustic device, that could be included within the acoustic device itself but when it is preferred not to do this due to packaging limitations or to encourage sterile procedures for use of the acoustic device.

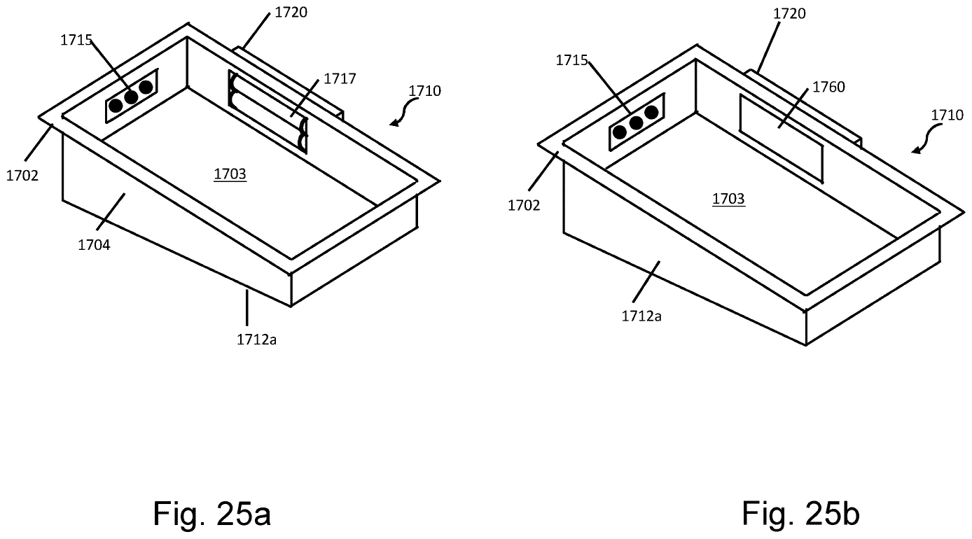

In one embodiment, as shown in FIG. 25(a), a bottom frame portion 1712a of two part cover 1710 similar to embodiments described above is provided with a battery for powering the wedge shaped acoustic device when inserted into cavity 1704 with a neat fit seated against acoustically transparent wall 1703. It will be understood that, alternatively or additionally, the top frame portion (not shown) may also be provided with battery power. Use of a battery powered cover has the object of ensuring use with an otherwise unpowered acoustic device.

Bottom frame portion 1712a is provided with an electric socket 1715 to which power is supplied from batteries 1717 stored in a battery compartment or pack 1720 which may be provided with a removable cover 1760 as shown in FIG. 25(b). A single battery could be used. In turn, power is supplied to the acoustic device when connected, through a complementary connecting plug, to electric socket 1715. It will be understood that the positions of socket and plug may be reversed, with the socket being provided on the acoustic device and the plug being provided in place of electric socket 1715. It will be further understood that cover 10 could be configured to plug into a mains or other power supply with electric socket 1715 communicating with that power supply through further socket(s), plug(s) or wiring.

Bottom frame portion 1712a also includes an overhanging brim 1702 to improve grip and usability.

FIG. 25(b) shows removable cover 1760, for battery compartment 1720 of bottom frame portion 1712a, in a closed position. The cover may be attached to side wall 1722 by any suitable fixing or connection means. Clip, screw, friction fit and slide mechanisms of fixing could be used.

In a further embodiment, the cover could be provided with means for increasing visibility of the screen of the acoustic device. For example, the cover could be, or could provide, a polarising filter for the acoustic device screen. The polarising filter acts to enhance the screen visibility of the acoustic device which may otherwise be difficult to observe. By enhancing screen visibility, the use of the cover is encouraged and may be ensured by requiring that the polarising filter or other screen visibility enhancing means be in position for the acoustic device screen to be visible to a user.

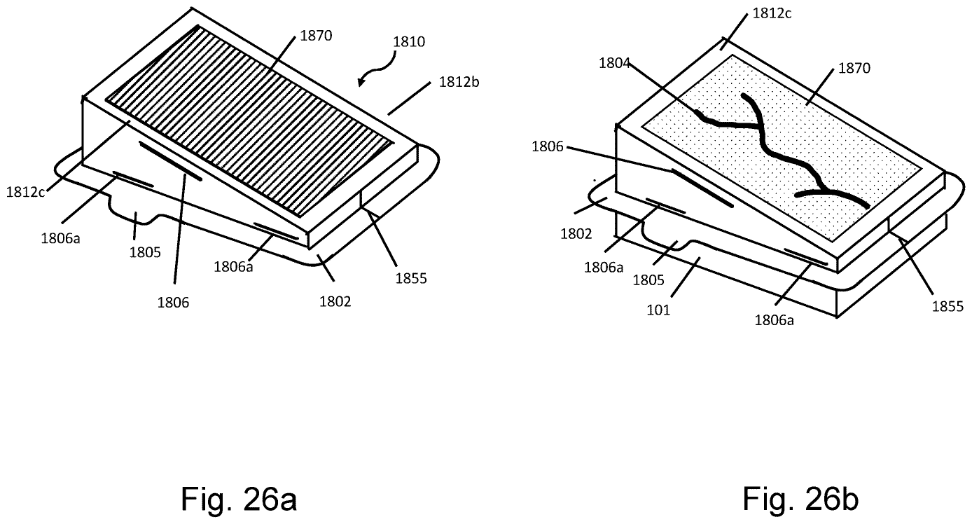

FIGS. 26(a) and 26(b) show the functionality of the polarising filter which makes visible the display screen of an acoustic device enclosed within the two part cover 1810 which has a rigid frame including top frame portion 1812b and is similar in configuration to those embodiments described above having both overhanging brim 1802 to ease grip, tab 1805 and centre mark 1855 for the acoustic device, again of wedged shape with a sloping wall providing enhanced viewability of its screen.

The polarising filter 1870 allows the ultrasonically derived image of a vein 1804 on the display screen to be viewed, for example during a cannulation procedure as shown in FIG. 26(b).

FIG. 26(a) shows top frame portion 1812b in the absence of the acoustic device. Top frame portion 1812b includes a polarising filter extending over substantially all of the upper surface 1812c. It will be understood that, while less preferred, the polarising filter 1870 could extend over a smaller area to allow viewing over a selected window of the display screen when in position.

FIG. 26(b) allows viewing of an image of vein 1804 as displayed on the display screen of the acoustic device 101 as neatly fitted within the top frame portion 1812b of two part cover 1810. It is to be understood that the polarising filter 1870 is not limited to horizontal or vertical grating patterns but may have any configuration necessary to achieve the viewability function.

The cover may be provided with an in-built screen to display patient information. The object of the in-built screen is to ensure that the cover is applied during all procedures using the acoustic device with sterility thus being maintained. Such a screen may or may not include touch screen functionality and/or buttons to control the display (e.g. brightness etc.). The in-built screen may also permit presentation of different displays such as subcutaneous structure mapping (for example 2D or 3D mapping of vessels), B-mode in the case of ultrasound imaging or any other patient data required during conduct of a procedure.

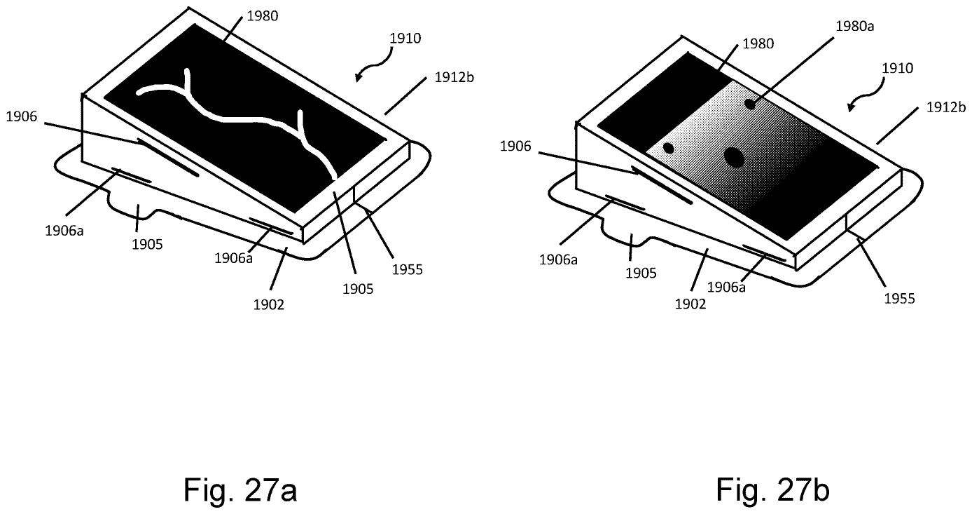

Such an embodiment of cover 1910 is shown in FIGS. 27(a) and 27(b). In-built screen 1980 is provided in the rigid top frame portion 1912b with the screen being interfaced, as known in the electronic arts, with the acoustic device 101 neatly fitted within it. In this embodiment, applied to ultrasonic imaging, in-built screen 1980 displays ultrasonic imaging based information to the user. A vein path is displayed on the in-built screen 1980 of FIG. 27(a) whereas the in-built screen 1980 of FIG. 27(b) displays ultrasound B-mode information 1980a. In-built screen 1980 is not limited to such displays and any required procedural information may be displayed.

Top frame portion 1912b is similar in configuration to those embodiments described above, including that shown in FIGS. 27(a) and 27(b), having both overhanging brim 1902 to ease grip, tab 1905 and centre mark 1955 for the acoustic device 101.

In embodiments of the cover of the invention, including covers as described above, the cover may optionally be configured to control the beam direction of the acoustic device. The cover may control beam direction by one or more of the modes of direct transmission, beam steering, beam bending, convergence of acoustic wave fronts and the cover may be configured accordingly. Such configuration may involve, without limitation, selection of orientation, shape, size or thickness of the cover or cover walls or surfaces provided on those walls, in particular a wall or surface proximate to a patient's skin.

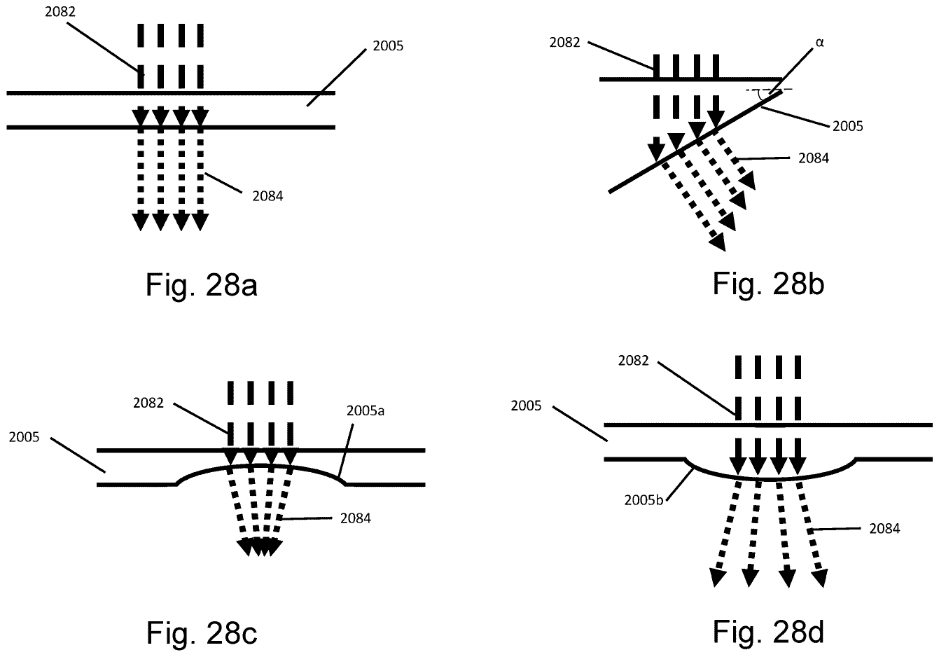

Referring to FIG. 28(a) to 28(d), there are shown embodiments for controlling direction or properties of an ultrasonic beam emitted from an acoustic device during use. Modes of control shown are respectively direct transmission, beam steering, convergence of acoustic wave fronts and divergence of acoustic wave fronts.

FIG. 28(a) illustrates the ability of a thin acoustically transparent membrane 2005 provided on a patient facing surface of embodiments of the cover, for example of configuration the same or similar to those described above, to allow for direct transmission of the incoming ultrasound beam 2082. Such a thin acoustically transparent membrane would have an acoustic impendence and speed of sound similar to human tissue to prevent the reflection of acoustic energy at the interface. The resultant directly transmitted beam 2084 travels in the same direction as the incoming ultrasound beam 2082.

FIG. 28(b) illustrates how incoming ultrasound beam 2082 may be steered by adjusting the orientation, i.e. the angle a to the horizontal, of the thin membrane 2005 provided on a patent facing surface of embodiments of the cover. Unlike the embodiment of FIG. 28(a), the transmitted ultrasound beam 2084 no longer travels in the same direction as incoming ultrasound beam 2082. It will be understood that beam steering can be achieved other than by adjusting orientation of the thin membrane 2005. The thin membrane 2005 may have other properties, for example refractive capability, that achieve the same result of changing an incoming ultrasound beam direction 2082 to a different direction transmitted ultrasound beam 2084.

FIG. 28(c) illustrates how incoming ultrasound beam 2082 may be converged by providing the thin membrane 2005 provided on a patient facing surface of embodiments of the cover with a selected degree of concavity 2005a. By converging the transmitted beam 2084, different focal depths can be achieved as desired by a user. Beam convergence may be provided by means other than providing concavity 2005a, for example by providing beam convergence through configuration of the material properties of thin membrane 2005.

FIG. 28(d) illustrates how incoming ultrasound beam 2082 may be diverged by providing the thin membrane 2005 provided on a patent facing surface of embodiments of the cover with a selected degree of convexity 2005b. By diverging the transmitted beam 2084, different focal depths can be achieved as desired by a user. Beam divergence may be provided by means other than providing convexity 2005b, for example by providing beam divergence through configuration of the material properties of thin membrane 2005.

It will be understood that, in other embodiments, control over beam direction may be exercised for energy other than ultrasound energy, for example infra-red energy.

A dispenser may be provided for dispensing covers of embodiments of the invention the same or similar to those described above. Such a dispenser desirably provides a sterile supply of covers ready for use though a clean supply of covers may be acceptable for some procedures in some countries. The dispenser may be provided with a loading means for directing covers to the dispenser. However, manual reloading of the dispenser is also possible in other embodiments.

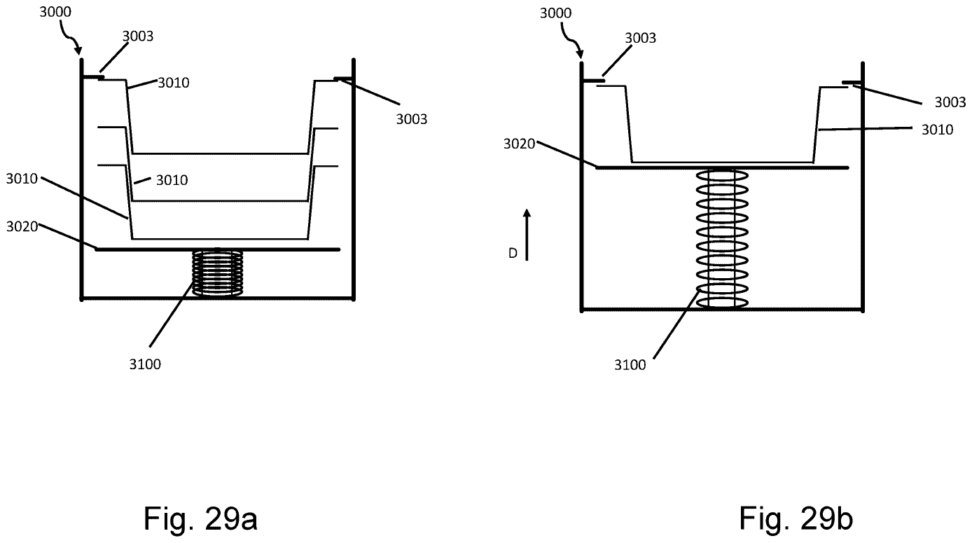

One embodiment of dispenser 3000 is shown in FIGS. 29(a) and 29(b). The dispenser 3000 includes a stage or platform 3020 for holding a supply 3005 of cover portions 3010 under sterile conditions or, alternatively, where further sterile packaging is used, the dispenser itself would not need to be sterile though it would desirably be clean. Covers 3010 have a similar configuration to the bottom frame portions described above and are able to form a nested arrangement within the dispenser 3000 as shown in FIG. 29(a). As, in this embodiment, cover portions are rectangular in plan the dispenser 3000 may likewise be rectangular in plan.

A fully loaded position of dispenser 3000 is schematically illustrated by FIG. 29(a). Stage 3020 moves upward or downward subject to the action of a spring 3100 which also provides support to stage 3020. Spring 3100 is biased to move in an upward direction ‘D’. As covers 3010 are removed from the dispenser 3000 reducing the downward load on the stage 3020 and the spring 3100, the spring 3020 is subject to less compression and moves upward as indicated by a comparison between FIGS. 29(a) and (b). The upward movement is sufficient to present a new cover 3010 for use by a user. Covers 3010 are also held in place by a locking plate or flange 3003 to, through compression on cover(s) 3010 present in the dispenser 3000, prevent premature release of covers 3010 from the dispenser 3000.

Dispenser 3000 allows an easy to use means for delivering covers 3010 to a user for ready application to an acoustic device, such as ultrasound device 101. While a spring loaded stage 3020 has been described above, it will be apparent that other embodiments could include an electrically or hydraulically operated stage 3020. Covers 3010 or frame portions could also be located in compartments of a rotating wheel.

While the above description has been made with reference to ultrasound probes, it will be understood that cover 10 (as well as the cover of other embodiments as described above) may be deployed to cover other acoustic medical devices, in particular those used for medical imaging. Acoustic devices for veterinary applications may also be covered by cover 10 (as well as the cover of other embodiments as described above).

Further, while the above description has been made with reference to a human patient, the cover 10 (as well as the cover of other embodiments as described above) may also be used to cover acoustic devices used for veterinary procedures.

### Applications

Cover 10 (as well as the cover of other embodiments as described above) is compatible with a wide range of acoustic medical procedures that requires an instrument where a sterile barrier between the instrument and a subject is required. Typically, the procedure would use a needle or needle like device like a catheter or cannula. The cover 10, by way of example and ease of description below), is not limited to any particular type of needle, cannular or catheter, e.g. from Becton Dickinson (BD), BBraun or Teleflex. The cover 10 may be used for applications including, but not limited to: peripheral venous cannulation, central venous cannulation, varicose veins, phlebotomy, venipuncture, surgical ultrasound applications, epidural procedures, steroidal administration, trauma on skin ultrasound and general ultrasound.

### Example 1

In a first example, cover 10 may be used for hand-held ultrasound guided peripheral venous cannulations where no particular expertise is needed by a single person who can perform the procedure in a sterile manner. The acoustic coupling medium, fastening mechanism and needle guide are comprised within the cover 10.

### Example 2

In a second example, the cover 10 may be used for other ultrasound guided cannulation applications including, but not limited to: central venous cannulations and intra-arterial cannulations. The cover 10 allows the procedure to be performed in a sterile manner by one person without the need for specific training. The acoustic coupling medium, fastening mechanism and needle guide are comprised within the cover 10.

### Example 3

In a third example, the cover 10 may be used in venipuncture for applications including medical testing, transfusion, donation or research. The acoustic coupling medium, fastening mechanism and needle guide are comprised within the cover 10. With the aid of an imaging device and the cover 10, the number of persons able to donate blood may be increased through the ability to show veins that could not previously be visualised or palpated.

### Example 4

In a fourth example, the cover 10 may be used for lumbar puncture and central neuraxial blocks including spinal, epidural, combined spinal-epidural and caudal epidural injections, particularly for subjects where anatomical landmarks are not visible (i.e. obesity, upper spinal levels). The cover is compatible with a wide range of ultrasound probe types, allowing the area of interest to be imaged in a sterile manner allowing real time needle guidance into the spine and spinal canal.

### Example 5

In a fifth example, the cover 10 may be used for injections of local anaesthetics or corticosteroids into joints, soft tissue or around tendons for inflammation reduction and pain relief. Real-time needle guidance into the affected area is possible.

### Example 6

In a sixth example, the cover 10 may be used for ultrasound guided needle biopsies to sample tumours including inter alia the breast, lymph nodes and liver. The cover 10 is compatible with a wide range of ultrasound probe types, allowing the area of interest to be imaged in a sterile manner allowing real time needle guidance into the affected area.

### Example 7

In a seventh example, the cover 10 may be used for general diagnostic purposes that require skin contact. This includes, but is not limited to: ultrasound scans of the abdominal organs, pelvic organs, cardiac region and foetal scans. The cover 10 may be used to ensure that the ultrasound probe 101 is separated by a sterile barrier when contacting the skin and reduces risk of infection if the ultrasound probe 101 is inefficiently cleaned between uses. The containment of an amount of ultrasound gel required for the procedure allows imaging to be undertaken without the need for additional ultrasound gel.

In summary, the cover and methods of using it have a number of advantages including one or more, though preferably all, of the following:
(1) The cover allows sealing of an acoustic medical device, such as an ultrasound probe, allowing it to be used in a sterile manner while avoiding prior art difficulties in sterilising or cleaning such devices.
(2) The cover can also be deployed in a sterile manner, conveniently by a single user, not necessarily expert in the operation of complex ultrasound equipment.
(3) The cover allows combination of the functions of sterility, image coupling, device fastening and/or tourniquet into a single product. This combination allows avoidance of the need for aggregation of multiple separate components (e.g. sterile cover, sterile ultrasound gel, fastening strap and tourniquet).
(4) The cover allows reduction of waste by eliminating the amount of packaging required for the above multiple separate components.
(5) The cover allows simplification of the user's workflow.

Modifications and variations to the cover and method of use of the cover will be apparent to skilled readers of this disclosure. Such modifications and variations are deemed within the scope of the present invention.

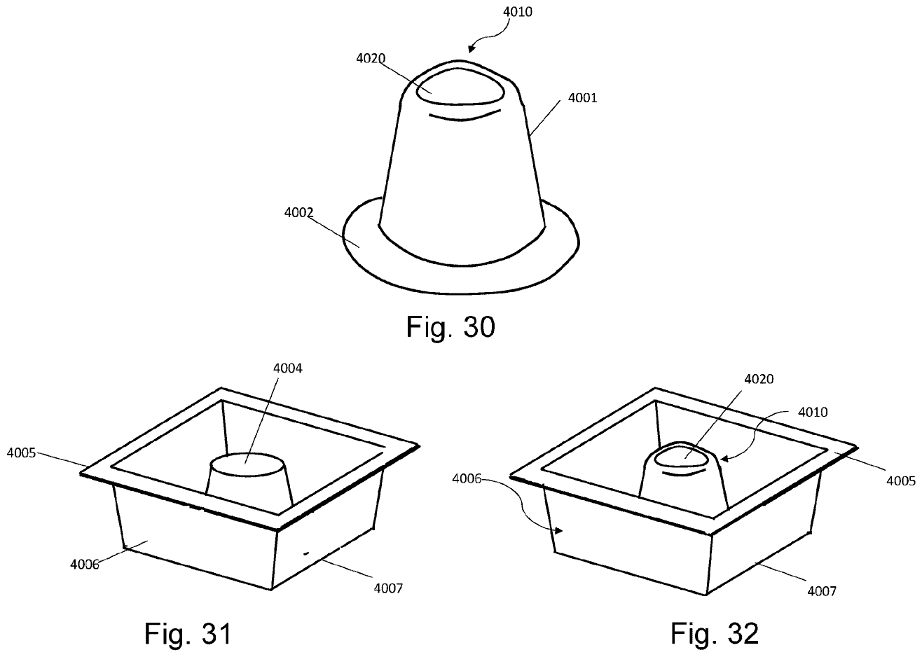

As shown in FIGS. 30 to 33, cover 4010 may be made in a form factor similar to a condom comprising an elongate elastic portion 4001 and a relatively inelastic or even rigid base portion 4002. Base portion 4002 may be circular, in the form of a ring, allowing a preferably tight fit to the acoustic device which cover 4010 is to enclose, for example ultrasonic device 101.

Cover 4010 is conveniently fabricated from an elastic polymer, such as natural or synthetic latex among other polymers. The material and diameter of the cover 4010 is such that the cover has sufficient flexibility to be stretched over an acoustic device, preferably with a single hand, to attach to the acoustic device with elastic force alone. As an example, the cover 1010 may have a diameter of less than 30 mm, more preferably less than 60 mm and most preferably a diameter of less than 100 mm.

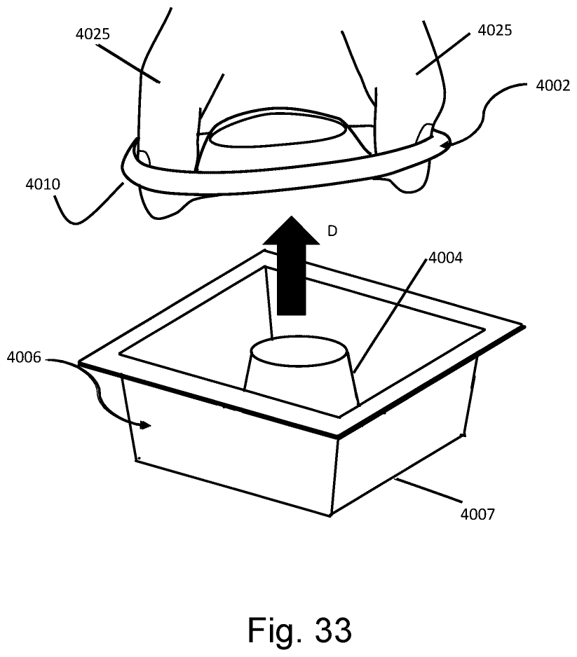

As shown in FIGS. 31 to 33, the cover 4010 may be packaged in a package such as a rigid receptacle 4006 having a base from which extends a vertical post 4004 containing an acoustic couplant 4020. Post 4004, preferably with a central location and here a truncated conical geometry, is aimed at easing the stretching of cover 4010 to cover the acoustic device. Cover 4010 may be rolled over the post 4004 and stretched up in direction D using one hand by the user with splayed fingers 4025 as shown in FIG. 33. Advantageously, the inside of the cover 4010 will be covered in acoustic couplant from the receptacle 4006. Rigid receptacle 4006 includes an overhanging brim 4005 to allow ease of grip.

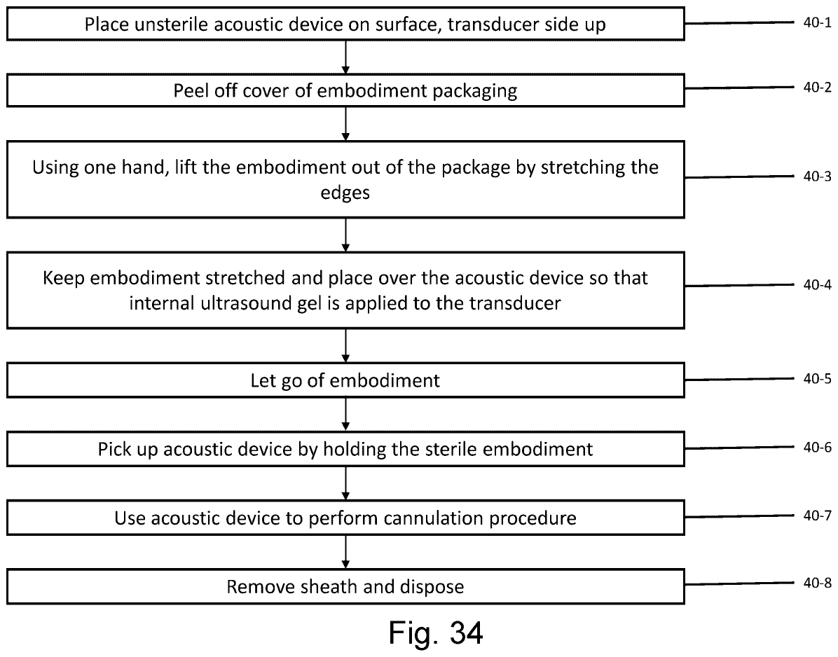

A workflow for use of cover 4010 is described below with reference to FIG. 34 with FIG. 35 allowing comparison with the workflow for two part cover embodiments, including two part cover 1210.

In step 40-1, the unsterile acoustic device, for example ultrasound device 101, is placed on a non-sterile surface, such as a table top, transducer side up.

In step 40-2, the sterile, packaged receptacle 4006 containing cover 4010 is opened by removing the packaging. Packaging for cover 1210 may be conventional medical packaging using either a polymer package or a box.

Steps 40-1 and 40-2 may be done in a non-sterile field but the following steps 40-3 to 40-8 must be conducted, for a procedure requiring sterility, in a sterile field. This can be achieved by applying sterile gloves and following other sterile techniques as known in the art of healthcare.

In step 40-3, cover 4010 is removed from receptacle 4006 by stretching the edges of cover 4010 and stretching it, conveniently with one hand, from post 4004.

In step 40-4, cover 4010 is stretched to cover ultrasonic device 101 (or other acoustic medical device) while ensuring that acoustic couplant is applied at least to the transducer side of ultrasonic device 101.

In step 40-5, the elasticity of cover 4010 allows it to closely fit ultrasound device 101.

In step 40-6, the ultrasound device 101-enclosed by cover 4010-is picked up for use in a sterile medical procedure, such as cannulation.

In step 40-7, covered ultrasound device 101 is used for the sterile medical procedure.

In step 40-8, the cover 4010 is removed and appropriately disposed of. Ultrasonic device 101 is re-used once covered with another cover such as 4010 or the two part cover as described above.

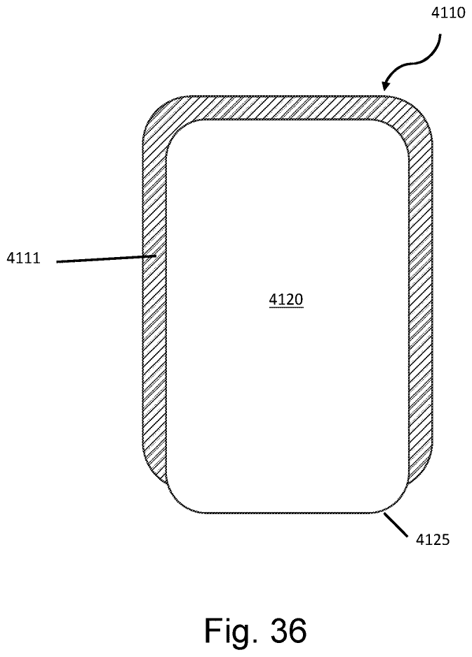

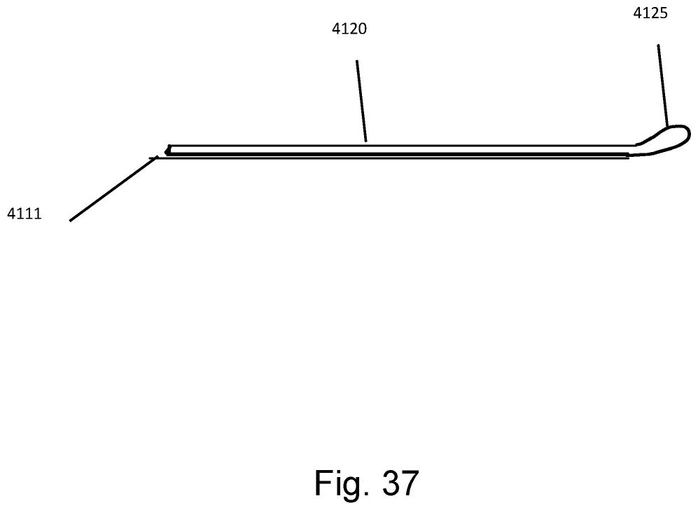

In another embodiment, as shown in FIGS. 36 and 37, the cover 4110 comprises an acoustically transparent dressing in the form of a pad. The pad may comprise a thin film 4111 which includes an acoustically transparent window forming a part or all of the pad 4110. The dressing may have any desired shape including as known in the art of medical dressings and including, without limitation, square, curved square, rectangular with a peelable backing 4120 being provided about the edge of the film. Removal of the peelable backing 4120 by pulling on tab 4125 exposes an adhesive area, conveniently running around the edge of the pad 4110. Advantageously, the film 4111 can then adhere to an acoustic device, such as ultrasound device 101, with the acoustically transparent window covering the imaging area providing ease in forming a barrier between the acoustic device and the patient. While this method is clean rather than sterile, it is useful for procedures that do not require a full sterile procedure, e.g. cannulation in certain countries. Conveniently, the dressing may adhere to skin, removing the need for applying an acoustic couplant between the patient and the dressing.

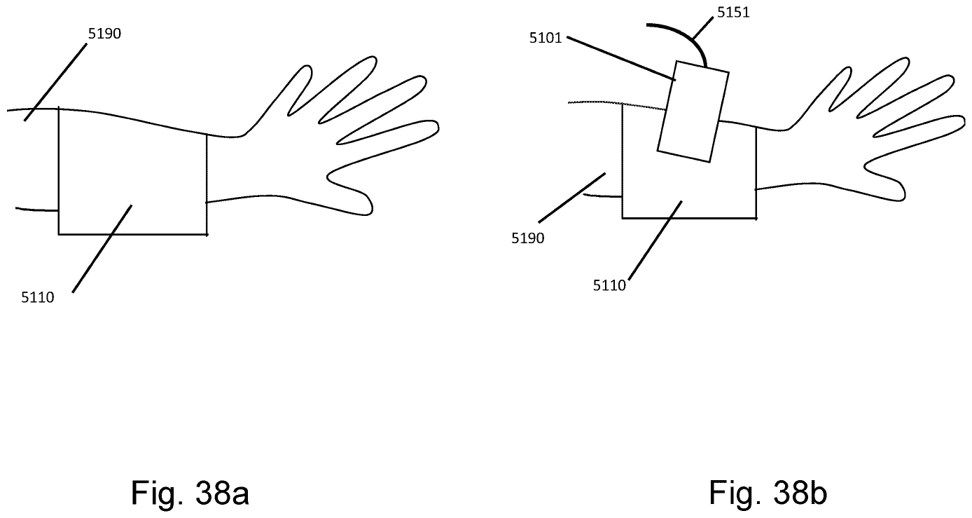

In another embodiment, shown in FIGS. 38(a) and 38(b), there is provided a sleeve 5110 for a patient body part, typically a limb such as arm 5190, which may be used with the cover of embodiments above or separately. The sleeve 5110 is intended to provide a sterile barrier for placement between the acoustic device 5101 and the patient's limb 5190 as conveniently shown in FIG. 38(b). Acoustic device 5101 is here supplied with power through cord 5151 rather than battery powered.

As such, the sleeve 5110 is conveniently provided in sterile packaging prior to removal for use with a patient. On removal from the sterile packaging, sleeve 5110 is wrapped around, draped around or otherwise placed on the patient's arm 5190.

The sleeve 5110 may be used throughout a procedure or only during a selected portion of the procedure, for example the period when a user is seeking vein identification, such as during a cannulation procedure. Again, it will be understood that the sleeve may be used for either medical or veterinary purposes.

Sleeve 5010—or any sleeve used in connection with the covers as described above—may include a pressure sensor and indicator which may conveniently indicate if the sleeve is excessively tight or excessively loose.

The sleeve may not include any acoustic couplant. Acoustic couplant would be applied separately both before and after placing it on a patient.

Throughout this specification, unless the context requires otherwise, the word “comprise” or variations such as “comprises” or “comprising”, will be understood to imply the inclusion of a stated integer or group of integers but not the exclusion of any other integer or group of integers.

1. (canceled)

2. A cover for an acoustic medical device comprising a frame including a volume defined by a base and side walls extending from the base for fitting a body of the acoustic medical device within the volume, said acoustic medical device being removable from said frame, wherein said frame includes an acoustically conductive portion and provides a sterile barrier between the acoustic medical device and a subject.

3. The cover of claim 1, wherein the side walls extend around all sides of the frame.

4. The cover of claim 3, wherein the side walls are provided as a shorter wall or as alignment guides extending from a plurality of sides of the base.

5. The cover of claim 3, wherein the frame comprises a plurality of portions, a first portion being the base or bottom frame portion which is connectable to a top the frame portion.

6. (canceled)

7. The cover of claim 2, wherein the frame is configured for frictional fitting of the acoustic medical device to the frame.

8. The cover of claim 5, wherein said frame portions are provided with gripping points to assist grip of the cover when an acoustic medical device is fitted within it.

9. The cover of claim 8, wherein said gripping points are selected from the group consisting of an overhanging brim for at least one frame portion and at least one groove.

10. (canceled)

11. The cover of claim 5, wherein portions of the cover have one or more flaps or tabs to facilitate fitting together of the portions of the cover.

12. (canceled)

13. The cover of claim 5, wherein the frame is fitted to the body of the acoustic medical device by frictional grip or adhesion.

14. The cover of claim 13, being conformable to the shape of the acoustic medical device but of slightly lesser dimension such that flexing of the frame is required to accommodate the acoustic medical device with a frictional grip retaining the body of the acoustic medical device within the frame.

15. The cover of claim 13, wherein the frame includes alignment guides for the acoustic medical device to facilitate friction fitting of the acoustic medical device within the frame.

16. The cover of claim 5, wherein the base of the frame accommodates an acoustic coupling medium for reducing acoustic impedance between the acoustic medical device and the closure, whereby quality of data collected by the acoustic medical device is improved.

17. The cover of claim 16, wherein the base of the frame comprises a compartment or pocket for containment of acoustic coupling medium.

18. (canceled)

19. The cover of claim 17, wherein the pocket contains the acoustic coupling medium to a specific area proximate the transducer array in an ultrasound probe.

20. The cover of claim 17, wherein the cover comprises the transducer array of an ultrasound probe.

21. The cover of claim 19, comprising a battery power supply for the acoustic medical device.

22. The cover of claim 19, comprising a screen for displaying data acquired by the acoustic medical device.

23. The cover of claim 17, wherein the pocket further comprises at least one removable cover in the form of a thin film or membrane comprising a plurality of pores to allow passage of excess acoustic coupling medium, said removable cover being peelable off the base to expose the acoustic coupling medium.

24. (canceled)

25. The cover of claim 23, wherein said removable cover has a tab.

26. (canceled)

27. The cover of claim 23, wherein each removable cover includes indicia indicating the order of removal of each removable cover.

28. The cover of claim 3, wherein the frame is low profile, covering less than 50% of the side.

29. The cover of claim 25, comprising a needle guide for guiding placement of a needle or needle-like object to be used in a medical procedure; and a connection means, whether connected to the frame or the removable closure, for detachably connecting the cover to a subject.

30. (canceled)

31. The cover of claim 14 wherein the acoustic medical device is a portable acoustic medical imaging device.

32-33. (canceled)
